(19) United States
(12) Patent Application Publication
(10) Pub. No.: US 2023/0235387 A1
(43) Pub. Date: Jul. 27, 2023

(54) DEVICES AND METHODS FOR GENOMIC STRUCTURAL ANALYSIS

(71) Applicants: DIMENSIONGEN, GRAND CAYMAN (KY); Dimension Genomics Inc., San Diego, CA (US)

(72) Inventors: Michael David Austin, San Diego, CA (US); William Ridgeway, San Diego, CA (US)

(73) Assignees: DIMENSIONGEN, GRAND CAYMAN (KY); Dimension Genomics Inc., San Diego, CA (US)

(21) Appl. No.: 18/001,773

(22) PCT Filed: Jun. 28, 2021
(86) PCT No.: PCT/US2021/039348

§ 371 Date: Dec. 14, 2022

Related U.S. Application Data
(60) Provisional application No. 63/046,069, filed on Jun. 30, 2020.
(60) Provisional application No. 63/143,857, filed on Jan. 31, 2021.

(51) Int. Cl. C12Q 1/6869; C12Q 1/6809; C12Q 1/6886

(52) U.S. Cl. CPC C12Q 1/6869; C12Q 1/6809; C12Q 1/6886

(57) ABSTRACT

Disclosed are methods for generating physical maps from feature density profiles of a nucleic acid using a constriction device, and associated methods of analyzing said genomic profiles. In addition, disclosed are devices and methods for analyzing secondary, tertiary and quaternary structures on nucleic acids in spatial and temporal context of the 3-D organization of the genome in a constriction or sensor device.

### CROSS-REFERENCE TO RELATED APPLICATIONS

This document claims the benefit of priority to U.S. Provisional Application Ser. No. 63/046,069, filed Jun. 30, 2020, and to U.S. Provisional Application Ser. No. 63/143,857 filed Jan. 31, 2021 each of which is hereby incorporated by reference in its entirety, and this document is the US regional phase entry of PCT/US2021/039348, filed Jun. 28, 2021, and published as WO2022/005957 on Jan. 6, 2022, which is hereby incorporated by reference in its entirety.

### BACKGROUND

Constriction (or nanopore) with nano-sized sensors have demonstrated to be capable in a wide range of applications. Such devices are the sources of much academic and commercial investigation, as they hold the promise of direct, ubiquitous and inexpensive bio-molecule analysis, in particular nucleic acid sequencing and mapping, in situ at single molecule and single cell level. For constriction-like devices, the typical operation involves translocating a polymeric molecule through a constriction, or passing by a detecting sensor and measuring an electrical signal that is modulated as the macromolecules or polymers translocate. The quality of the signal generated is influenced by many factors, including the constriction size, physical size and shape of the constriction and surrounding regions, the translocation speed, and the physical size, feature characteristics contrast of the entities along the polymer that are being detected, to name but a few. For sequencing applications where-by the goal is to elucidate individual nucleotides, the technical challenges are substantial. In order to overcome such challenges, different techniques have been pursued such as reading short units of nucleotides (kmers) rather than individual nucleotides [Reid, 2012, patent application], or modifying the nucleotides themselves to increase their relative contrast with each other [Gundlach, 2013, patent application]. However, even with these improvements, challenges remain, and thus different applications have been pursued that allow for less stringency on the constriction device specifications and operation. These include molecule identification via binding of specific labels, and physical mapping via binding of sequence specific labels along the molecule. In both cases, the size contrast between the nucleic acid and the label(s) provides a stronger signal than single nucleotide variation along the polymer itself. As such, generally such applications allow for larger constrictions with associated larger constriction size variation and/or for faster translocation speeds, which ultimately can lead to higher throughput and/or lower cost per run.

As disease-association studies, clinical genetic testing, and various data banks, have grown with the increased accessibility of next generation genome sequencing, our knowledge of medically relevant genetic mutations of the population is built largely around the interpretation of single-nucleotide variants (SNVs). However, Structural variants (SVs) rearrange large segments of DNA and can have profound consequences in evolution and human disease [Perry, 2008], [Weischenfeldt, 2013]. In one recent study of SVs constructed from 14,891 genomes across diverse global populations (54% non-European), a rich and complex landscape of 433,371 SVs were discovered from which SVs are estimated to be responsible for 25-29% of all rare protein-truncating events per genome, that is detrimental or with biological consequences [Collins, 2020].

Physical mapping techniques have proven to be highly effective either by themselves, or in conjunction with sequencing technologies, to elucidate complicated genomic features that typically span over large ranges (>10 kbp) [Bocklandt, 2019], which are often difficult to be spanned and resolved with sequencing alone. Furthermore, the complicated primary, secondary, tertiary and quaternary structures in which portions of DNA take on within the cell to be ‘functional’, are but lost during sequencing or conventional optical genome mapping. These structures must be inferred via the underlying sequence or insertion of barcodes [Szabo, 2019]. These methods of structure analysis use a “bottom up” approach of isolating and breaking up these discrete or semi-discrete segments and domains of genome for interrogation, and then assemble them back using certain hypothesis and assumptions within the mathematical model. However, a “top down” direct physical mapping of the location, spatial positions, and dynamic interactive processes of these functional components and complexes within genome sequence, chromatin, chromosomes, and nucleus, especially in their contiguous or continuous native context without physical disruption, would be immensely valuable in elucidating these biologically important structures in an efficient manner, and will help further our understanding of genetics and etiology of diseases including many rare and undiagnosed disorders and cancer.

It is well established that discrete and distant genomic sequence elements could regulate gene function over long distance (https://www.genome.gov/Funded-Programs-Projects/ENCODE-Project-ENCyclopedia-Of-DNA-Elements). In recent years, it has become evident that the spatial organization of the genome is key for its function. How genome regulates its functions is associated with not only the primary level of linear sequence information, but also physical configurations in which the genome resides. How sequence elements and other cellular components interact with each other in cis or trans in a spatial and temporal fashion impacts how they function. Mammalian genomes are spatially organized into subnuclear compartments, territories, high order folding complexes, topologically associating domains (TADs), and loops to facilitate gene regulation and other important chromosomal functions such as replications. These structures are likely a source for many aberrant genomic recombination and errors with pathological consequences or biological impacts. It has been proposed that chromosomal territories, compartments, topologically associating domains (TAD), chromatin loop and local direct regulatory factors binding, bending and kinks of the genomic DNA polymers are regulated in a complex and sophisticated manner involving many nuclear and cellular components such as transcription factors, repressors, insulators, transactivators and enzymes. How exactly these 3-dimensional territories, compartments, TADs, and loops are generated or regulated is still under intensive investigation and unclear. Technologies able to directly visualize and map these intricate dynamic interactions in their native genomic, subcellular and subnuclear context would be extremely valuable for understanding how the primary sequencing information links with the 3-D organization of the genome, and thus contribute to a better understanding and characterization of the regulation of genes and ultimately end point biological and pathophysiological functions and consequences.

Here we present new devices and methods for using constriction or detecting sensor devices to generate nucleic acid physical maps, and to analyze nucleic acid primary, secondary, tertiary and quaternary structures and their associations.

### SUMMARY OF THE INVENTION

Disclosed are methods for generating feature density profiles, a type of linear physical map, of a nucleic acid molecule using a constriction device, and associated methods of analyzing said genomic profiles. For example, the local ratio of AT:CG base pairs within an arbitrary section of nucleic acid can vary between sections, such that the variation of this ratio along the length of a nucleic acid can provide a unique signature, much like the underlying sequence of base pairs, and thus providing linear physical map which can be used to identify and compare the nucleic acid molecule or sections therein to a reference. This profile could potentially provide insight of genomic variations such as pathological deletions and insertions, genomic rearrangements over much longer range of genomic regions then what are typically achievable by sequencing methods. It is well established that these large genomic features at the structural level could impact genomic functions.

Further disclosed are methods and devices for analyzing physical structures in nucleic acid molecules.

Aspects of the present disclosure include a method for analyzing a long nucleic acid molecule, comprising: (a) partially de-naturing at least a portion of said long nucleic acid molecule by exposing at least a portion of the molecule to at least one denaturing condition; (b) translocating at least a portion of said long nucleic acid molecule between a first conductive liquid medium and a second conductive liquid medium through at least one constriction region of at least one constriction device; (c) interrogating at least one signal associated with the at least one constriction device as the nucleic acid molecule interacts with the at least one constriction region of said at least one constriction device; and (d) determining a binned denaturing profile along at least a portion of the long nucleic acid molecule from said at least one signal.

In some embodiments, an ion current through the constriction region is measured to generate the signal.

In some embodiments, the at least one constriction device comprises an electrode gap of sufficient proximity to the constriction region of the device such that the long nucleic acid molecule translocating through said constriction region also translocates between said electrode gap, such that an electrical measurement can be performed to generate the signal.

In some embodiments, the at least one constriction device comprises a sensor of sufficient proximity to the constriction region of the device such that said molecule translocating through said constriction region will be sensed by the sensor, generating the signal.

In some embodiments, the sensor comprises a transistor.

In some embodiments, the sensor comprises a functionalized surface.

In some embodiments, the constriction of the constriction device is tangible.

In some embodiments, the constriction of the constriction device is intangible.

In some embodiments, the signal is captured in the constriction region of the constriction device.

In some embodiments, the signal is captured in proximity to the constriction region of the constriction device.

In some embodiments, the signal generated from the portion of the partially melted long nucleic acid molecule is measurably different than a signal that would have resulted from the same portion of said molecule in a fully hybridized state.

In some embodiments, the denaturing condition comprises a temperature.

In some embodiments, the denaturing condition comprises a reagent.

In some embodiments, the denaturing condition comprises an ionic strength.

In some embodiments, the denaturing condition comprises a pH.

In some embodiments, the denaturing condition is modulated.

In some embodiments, the denaturing condition is modulated during the interrogation.

In some embodiments, the denaturing condition is modulated between multiple interrogation events of said molecule.

In some embodiments, the denaturing condition is modulated to increase uniqueness of the binned denaturation profile of at least a portion of said long nucleic acid molecule.

In some embodiments, the modulation is controlled by a feedback system in which at least one input parameter is the signal from said constriction device.

In some embodiments, a first side of the constriction region has a first denaturing condition and a second side of the constriction region has a second denaturing condition, and wherein the first denaturing condition and the second denaturing condition are different.

In some embodiments, at least a portion of said long nucleic acid molecule is interrogated by said constriction device a plurality of time.

In some embodiments, said plurality of interrogations are used to generate a consensus binned denaturation profile.

In some embodiments, the binned denaturation profile constitutes a linear physical map.

In some embodiments, comprising comparing said linear physical map to a reference.

In some embodiments, a variation relative to said reference indicates a structural variation in the long nucleic acid molecule relative to the reference.

In some embodiments, said comparing is used to identify information associated with a disease.

In some embodiments, this comparing is used to identify at least a portion of the long nucleic acid molecule.

In some embodiments, identifying the at least a portion of the long nucleic acid molecule comprises assigning an originating organism, class, species, ethnicity, family genealogy, individuals, tissues, cells, chromosome, phase, variant, gene, or location within a genome to the long nucleic acid molecule.

Aspects of the present disclosure include a method for analyzing higher order nucleic acid structure of a long nucleic acid molecule, comprising: (a) translocating at least a portion of said long nucleic acid molecule between a first conductive liquid medium and a second conductive liquid medium through at least one constriction region of at least one constriction device; (b) interrogating at least one signal associated with the at least one constriction device as the long nucleic acid molecule translocates through the at least one constriction region of said at least one constriction device; and (c) determining a property of said structure from said at least one signal.

In some embodiments, an ion current through said constriction region is measured to generate the signal.

In some embodiments, the at least one constriction device comprises an electrode gap in proximity to the constriction region such that the long nucleic acid molecule translocating through said constriction region will also translocate through said electrode gap, such that an electrical measurement can be performed to generate the signal.

In some embodiments, the at least one constriction device comprises a sensor of sufficient proximity to said device's constriction region, such that said long nucleic acid molecule translocating through said constriction region will be sensed by the sensor, generating the signal.

In some embodiments, the sensor comprises a transistor.

In some embodiments, the sensor comprises a functionalized surface.

In some embodiments, the constriction of the constriction device is tangible.

In some embodiments, the constriction of the constriction device is intangible.

In some embodiments, the signal is captured in the constriction region of the constriction device.

In some embodiments, the signal is captured in proximity to the constriction region of the constriction device.

In some embodiments, the signal generated from the portion of the long nucleic acid molecule with a structure is measurably different than a signal that would have resulted from the same portion of said molecule without said structure.

In some embodiments, the higher order nucleic acid structure comprises a nucleosome.

In some embodiments, the higher order nucleic acid structure comprises a nucleosome clutch.

In some embodiments, the higher order nucleic acid structure comprises chromatin.

In some embodiments, the higher order nucleic acid structure comprises a chromatin nanodomain.

In some embodiments, the higher order nucleic acid structure comprises a CCCTC binding factor.

In some embodiments, the higher order nucleic acid structure comprises a loop.

In some embodiments, the higher order nucleic acid structure comprises a topologically associating domain.

In some embodiments, the higher order nucleic acid structure comprises a loop domain.

In some embodiments, the higher order nucleic acid structure comprises a compartment A.

In some embodiments, the higher order nucleic acid structure comprises a compartment B.

In some embodiments, the higher order nucleic acid structure comprises an enhancer-promoter complex.

In some embodiments, the higher order nucleic acid structure comprises an insulator complex.

In some embodiments, the higher order nucleic acid structure comprises a transcription factor complex.

In some embodiments, the higher order nucleic acid structure comprises a CTCF protein.

In some embodiments, the higher order nucleic acid structure comprises a PDS5 protein.

In some embodiments, the higher order nucleic acid structure comprises a WAPL protein.

In some embodiments, the higher order nucleic acid structure comprises a heterochromatin, a euchromatin, or a heterochromatin-euchromatin boundary.

In some embodiments, the higher order nucleic acid structure comprises a transcription factor.

In some embodiments, the higher order nucleic acid structure comprises a methyl-binding protein.

In some embodiments, the higher order nucleic acid structure comprises a chromatin remodeling protein.

In some embodiments, the higher order nucleic acid structure comprises a Histone deacetylase (HDAC).

In some embodiments, the higher order nucleic acid structure comprises a nucleic acid binding protein.

In some embodiments, the higher order nucleic acid structure comprises a regulatory factor binding protein.

In some embodiments, the higher order nucleic acid structure comprises a nucleic acid repair protein.

In some embodiments, the higher order nucleic acid structure comprises a telomere modification protein.

In some embodiments, the higher order nucleic acid structure comprises a repeat region binding protein.

In some embodiments, the higher order nucleic acid structure comprises a ribonucleic acid (RNA), small interfering RNA (siRNA), micro RNA (miRNA), guide RNA (gRNA), Long non-coding RNA (lncRNA).

In some embodiments, the higher order nucleic acid structure comprises a nucleoprotein complex.

In some embodiments, the higher order nucleic acid structure comprises a CRISPR Cas9 complex.

In some embodiments, the higher order nucleic acid structure comprises an argonaut complex.

In some embodiments, the higher order nucleic acid structure comprises a cohesin associated loop.

In some embodiments, the higher order nucleic acid structure comprises a condensin associated loop

In some embodiments, at least one sequence-specific labeling body is bound to said long nucleic acid molecule.

In some embodiments, the property of the said structure comprises information associated with a disease.

In some embodiments, the disease is a cancer.

In some embodiments, the property of said structure comprises physical size of the structure.

In some embodiments, the property of said structure comprises physical orientation with respect to a long axis of said long nucleic acid molecule.

In some embodiments, the property of said structure comprises flexibility of the structure.

In some embodiments, the property of said structure comprises a number of loops contained within.

In some embodiments, the property of said structure comprises a length of at least one loop contained within.

In some embodiments, the property of said structure is interrogated using at least two different translocation forces.

In some embodiments, the property of said structure is interrogated using at least two fluidically connected constriction devices, each having a different constriction region property.

In some embodiments, the constriction region property comprises a cross-section.

In some embodiments, the constriction region property comprises a critical dimension.

In some embodiments, the constriction region property comprises a baseline un-occupied measured constriction device signal for fixed measurement condition.

In some embodiments, the constriction region property comprises a baseline measured constriction device signal when interrogating a known control molecule or macromolecule.

In some embodiments, the constriction region property comprises a surface energy.

In some embodiments, the constriction region property comprises translocation length.

In some embodiments, the constriction region property comprises surface functionalization.

In some embodiments, a selection mechanism is used to determine the order in which the at least two constriction devices will be used for interrogation.

In some embodiments, a selection mechanism is at least partially based a previous interrogation of said molecule.

In some embodiments, a selection mechanism is at least partially based on a constriction region property.

In some embodiments, the minimum translocation force on said long nucleic acid molecule necessary to translocate said molecule through said two constriction devices is different.

In some embodiments, a property of the solution fluidically connecting the two constriction devices can be modified while the long nucleic acid is in contact with the solution.

In some embodiments, the property comprises a reagent concentration.

In some embodiments, the reagent is a digestive enzyme.

In some embodiments, the property comprises an ionic concentration.

In some embodiments, the property comprises a pH, a conductivity, a density, or a viscosity.

In some embodiments, the modification of the solution property is used to modify the physical conformation of said higher order nucleic acid structure.

In some embodiments, the long nucleic acid molecule is bound with at least two labeling bodies of one label body type.

In some embodiments, the said labeling bodies constitute a physical map.

In some embodiments, said labelling bodies can be interrogated by said constriction device.

In some embodiments, said labelling bodies can be interrogated by a fluorescent interrogation device.

In some embodiments, the fluorescent interrogation is done while at least a portion of said long nucleic acid molecule is being interrogated by at least one of the at least two constriction devices.

In some embodiments, the long nucleic molecule is at least partially in a partially melted state while being interrogated by one of the at least two constriction devices.

In some embodiments, said partially melted state constitutes a physical map.

In some embodiments, said physical map is compared to a reference.

Aspects of the present disclosure include a constriction device comprising a constriction region having a first side and a second side, wherein a retarding force can be applied on a long nucleic acid molecule at the first side that opposes a translocation force applied on said molecule while said molecule is translocating said constriction region of said constriction device.

In some embodiments, an ion current through said constriction region can be measured to generate a signal.

In some embodiments, the at least one constriction device comprises an electrode gap in proximity to the constriction region such that the long nucleic acid molecule translocating through said constriction region will also translocate through said electrode gap, such that an electrical measurement can be performed to generate a signal.

In some embodiments, the at least one constriction device comprises a sensor of sufficient proximity to said device's constriction region, such that said long nucleic acid molecule translocating through said constriction region will be sensed by the sensor, generating a signal.

In some embodiments, the sensor comprises a transistor.

In some embodiments, the sensor comprises a functionalized surface.

In some embodiments, the constriction of the constriction device is tangible.

In some embodiments, the constriction of the constriction device is intangible.

In some embodiments, the signal is captured in the constriction region of the constriction device.

In some embodiments, the signal is captured in proximity to the constriction region of the constriction device.

In some embodiments, the retarding force comprises a shear force.

In some embodiments, the shear force originates from an interaction between said long nucleic acid molecule and a fluid flow.

In some embodiments, the retarding force comprises a frictional force.

In some embodiments, the frictional force originates from an interaction between said long nucleic acid molecule and at least one fluidic feature.

In some embodiments, the fluidic feature comprises a patterned fluidic feature.

In some embodiments, the patterned fluidic feature comprises a pillar, a corner, a channel, a pit, a functionalized surface, a well, or a topological change.

In some embodiments, the fluidic feature comprises a porous material.

In some embodiments, the fluidic feature comprises a bead.

Aspects of the present disclosure include a device comprising a long nucleic acid molecule juxtaposed in a constriction region, wherein the constriction region separates a first side on which a retarding force is applied to the long nucleic acid molecule, from a second side on which a translocation force is applied to the long nucleic molecule.

In some embodiments, the first side comprises a first solution having a first ionic concentration, and the second side comprises a second solution having a second ionic concentration.

In some embodiments, the long nucleic acid exhibits differential base pairing strength in the first solution relative to the second solution.

In some embodiments, the long nucleic acid is at least partially denatured in the second solution.

In some embodiments, the long nucleic acid is labeled using a first label moiety.

In some embodiments, the first label moiety differentially binds to single stranded nucleic acids.

In some embodiments, the first label moiety differentially binds to double stranded nucleic acids.

In some embodiments, the first label moiety differentially binds to AT-rich nucleic acids.

In some embodiments, the first label moiety differentially binds to GC-rich nucleic acids.

In some embodiments, the first label moiety differentially binds to a specific nucleic acid sequence target.

In some embodiments, the first label moiety differentially binds to a chromatin moiety.

In some embodiments, the long nucleic acid molecule comprises chromatin.

In some embodiments, the long nucleic acid molecule comprises at least one nucleosome.

In some embodiments, the long nucleic acid molecule comprises at least one nucleosome clutch.

In some embodiments, the long nucleic acid molecule comprises a transcription factor.

In some embodiments, the long nucleic acid molecule is labeled using a second label moiety, wherein the first label moiety emits a first signal and wherein the second label moiety emits a second signal.

In some embodiments, the first label moiety exhibits a first binding specificity and the second label moiety exhibits a second binding specificity.

In some embodiments, the first binding specificity and the second binding specificity are different.

In some embodiments, the device comprises a monitoring moiety capable of detecting the first signal.

In some embodiments, the device comprises a monitoring moiety capable of detecting the first signal and the second signal.

In some embodiments, the device comprises an electrode gap in proximity to the constriction region, such that the electrode gap measures a property of the long nucleic acid molecule.

In some embodiments, the device comprises a sensor in proximity to the constriction region, such that the sensor measures a property of the long nucleic acid molecule.

In some embodiments, the monitoring moiety generates a first linear record of the first signal that corresponds to positioning of the first label moiety on the long nucleic acid molecule.

In some embodiments, the monitoring moiety generates a first linear record of the first signal that corresponds to the first label moiety on the long nucleic acid molecule at a first time point, and a second linear record of the second signal that corresponds to the second label moiety on the long nucleic acid molecule at a second time point.

In some embodiments, the first linear record at least partially maps to a reference, wherein the reference represents a linear record of a known nucleic acid.

In some embodiments, correlation of the first linear record to the reference indicates identity of at least a portion of the long nucleic acid molecule.

In some embodiments, identity indicates an originating organism, class, species, ethnicity, family genealogy, individuals, tissues, cells, chromosome, or location within a genome of the long nucleic acid molecule.

In some embodiments, a difference in correlation of the first linear record to the reference indicates a difference between the long nucleic acid molecule and the reference.

In some embodiments, the difference indicates a nucleic acid encoded disorder.

In some embodiments, the difference indicates a structural change in the long nucleic acid relative to the reference.

In some embodiments, the difference indicates a translocation in the long nucleic acid molecule.

In some embodiments, the difference indicates an insertion in the long nucleic acid molecule.

In some embodiments, the difference indicates a duplication in the long nucleic acid molecule.

In some embodiments, the difference indicates a deletion in the long nucleic acid molecule.

In some embodiments, the difference indicates cancer.

Aspects of the present disclosure include a method for analyzing a long nucleic acid molecule, comprising: (a) labelling at least a portion of said long nucleic acid molecule using at least two labelling bodies of at least one labeling body type to form a labeled portion of the long nucleic acid molecule, such that labeling body density of the at least one labeling body type along said long nucleic acid molecule corresponds to at least one feature of said long nucleic acid molecule; (b) translocating at least the labeled portion of said long nucleic acid through a constriction region of at least one constriction device, wherein the constriction region separates a first conductive liquid medium and a second conductive liquid medium; (c) interrogating at least one signal associated with the labeled portion of said long nucleic acid molecule as it translocates through the constriction region of the constriction device, wherein the signal at least partially comprises a contribution of at least one of the at least two labeling bodies; (d) using the at least one signal associated with the labeled portion of said long nucleic acid molecule to assign a binned labeling body density profile to at least the labeled portion of said long nucleic acid.

In some embodiments, an ion current through the constriction region is measured to generate the signal.

In some embodiments, the at least one constriction device comprises an electrode gap in proximity to the constriction region such that the long nucleic acid molecule translocating through said constriction region will also translocate through said electrode gap, such that an electrical measurement can be performed to generate the signal.

In some embodiments, the at least one constriction device comprises a sensor of sufficient proximity to said device's constriction region, such that said long nucleic acid molecule translocating through said constriction region will be sensed by the sensor, generating the signal.

In some embodiments, the sensor comprises a transistor.

In some embodiments, the sensor comprises a functionalized surface.

In some embodiments, the constriction of the constriction device is tangible.

In some embodiments, the constriction of the constriction device is intangible.

In some embodiments, the signal is captured in the constriction region of the constriction device.

In some embodiments, the signal is captured in proximity to the constriction region of the constriction device.

In some embodiments, the signal generated from the portion of the labelled long nucleic acid molecule is measurably different than a signal that would have resulted from the same portion of said molecule without said bound labelling body.

In some embodiments, the labelling body density positively correlates to a feature density of the long nucleic acid molecule.

In some embodiments, the labelling body density negatively correlates to a feature density of the long nucleic acid molecule.

In some embodiments, the feature comprises a denatured nucleotide pair.

In some embodiments, the feature comprises a hybridized nucleotide pair.

In some embodiments, the feature comprises an AT base-pair.

In some embodiments, the feature comprises an AT rich region.

In some embodiments, the feature comprises a CG base-pair.

In some embodiments, the feature comprises a CG rich region.

In some embodiments, the feature comprises an AU base-pair.

In some embodiments, the feature comprises an AU rich region.

In some embodiments, the feature comprises a methylated nucleotide.

In some embodiments, the feature comprises a sequence of at least 2 nucleotides.

In some embodiments, the feature comprises a sequence of no more than 2 nucleotides.

In some embodiments, the feature comprises a sequence of at least 3 nucleotides.

In some embodiments, the feature comprises a sequence of no more than 3 nucleotides.

In some embodiments, the feature comprises a sequence of at least 4 nucleotides.

In some embodiments, the feature comprises a sequence of no more than 4 nucleotides.

In some embodiments, the feature comprises a sequence of at least 5 nucleotides.

In some embodiments, the feature comprises a sequence of no more than 5 nucleotides.

In some embodiments, the feature comprises a sequence of at least 6 nucleotides.

In some embodiments, the feature comprises a sequence of no more than 6 nucleotides.

In some embodiments, the feature comprises a higher order nucleic acid structure.

In some embodiments, the feature comprises a histone.

In some embodiments, the feature comprises a nucleosome.

In some embodiments, the feature comprises a topologically associated domain.

In some embodiments, the feature comprises a DNA binding protein.

In some embodiments, the feature is a feature of any of the previously mentioned features, and wherein the signal indicates absence of the feature.

In some embodiments, the at least one labeling body type is fluorescent.

In some embodiments, the bin size is at least 5 nm.

In some embodiments, the bin size is at least 15 bp.

In some embodiments, the bin size is at least 10 nm.

In some embodiments, the bin size is at least 30 bp.

In some embodiments, the bin size is at least 50 nm.

In some embodiments, the bin size is at least 150 bp.

In some embodiments, the bin size is no more than 5 nm.

In some embodiments, the bin size is no more than 15 bp.

In some embodiments, the bin size is no more than 10 nm.

In some embodiments, the bin size is no more than 30 bp.

In some embodiments, the bin size is no more than 50 nm.

In some embodiments, the bin size is no more than 150 bp.

In some embodiments, the labeling body type binds to double-strand nucleic acid, and wherein said long nucleic acid molecule is at least partially denatured.

In some embodiments, comprising at least partially denaturing the long nucleic acid molecule.

In some embodiments, the labeling body type binds to single-strand nucleic acid, and wherein said long nucleic acid molecule is at least partially denatured.

In some embodiments, comprising at least partially denaturing the long nucleic acid molecule.

In some embodiments, the labeling body type specifically binds to AT-rich regions.

In some embodiments, the labeling body type specifically binds to CG-rich regions.

In some embodiments, comprising labeling at least a portion of said long nucleic acid molecule using at least two labelling bodies of a second labeling body type, wherein the at least one labeling body type associates with a first feature, and wherein the second labeling body type associates with a second feature.

In some embodiments, the second labeling body type makes a contribution to the signal that is distinct from a contribution of the first labeling body type to the signal.

In some embodiments, the at least one labeling body type is associated with a first feature, and wherein the second labeling body type is associated with absence of said feature.

In some embodiments, the second labeling body type makes a contribution to the signal that is distinct from a contribution of the first labeling body type to the signal.

In some embodiments, the at least one labeling body type is bound to the long nucleic while the long nucleic acid molecule is in a state of at least partial denaturation.

In some embodiments, the binned labeling body density profile delineates a linear physical map.

In some embodiments, said linear physical map is compared to a reference.

In some embodiments, a variation relative to said reference indicates a structural variation in the long nucleic acid molecule relative to the reference.

In some embodiments, a variation relative to said reference indicates information associated with a disease.

In some embodiments, comparison to the reference identifies at least a portion of the long nucleic acid molecule.

In some embodiments, comparison to the reference indicates an originating organism, class, species, ethnicity, family genealogy, individuals, tissues, cells, chromosome, phase, variant, gene, or location within a genome of the long nucleic acid molecule.

In some embodiments, at least a portion of said long nucleic acid molecule is interrogated by said constriction device a plurality of times to generate a plurality of interrogations.

In some embodiments, the plurality of interrogations are used to generate a consensus binned labeling body density profile.

In some embodiments, measuring at least one signal associated with the labeled portion of said long nucleic acid molecule comprises fluorescent interrogation.

In some embodiments, said fluorescent interrogation is performed while the long nucleic acid molecule is being interrogated by the constriction device.

In some embodiments, the fluorescent interrogation results in fluorescent data comprising spatial content of at least a portion of the long nucleic acid molecule's position within the constriction device at a certain time point, and wherein the fluorescent data is associated with constriction device data at the same time point.

In some embodiments, there is an association with at least a portion of data generated from the fluorescent interrogation and at least a portion of the signal.

In some embodiments, said fluorescent interrogation is used to generate a linear physical map of at least a portion of the long nucleic acid molecule.

In some embodiments, said physical map is compared to a reference.

In some embodiments, said fluorescent interrogation is used to determine information comprising a local stretch, global stretch, local velocity, or global velocity of the long nucleic acid molecule.

In some embodiments, said information is used in a feedback system to control said long nucleic acid molecule's translocation through the constriction device.

In some embodiments, the binned labeling body density profile is analyzed in a frequency domain.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The terms used in this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that way. Consequently, alternative language and synonyms may the same thing can typically be described in more than one be used for any one or more of the terms discussed here. Synonyms for certain terms are provided. However, a recital of one or more synonyms does not exclude the use of other synonyms, nor is any special significance to be placed upon whether or not a term is elaborated or discussed herein. All publications, patent applications, patents, and other references mentioned herein are incorporated by reference. In the case of conflict, the present specification, including definitions, will control. In addition, the materials, methods, and examples are illustrative only and are not intended to be limiting.

The invention is also described by means of particular examples. However, the use of such examples anywhere in the specification, including examples of any terms discussed herein, is illustrative only and in no way limits the scope and meaning of the invention or of any exemplified term. Likewise, the invention is not limited to any particular embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

For all drawings, the use of roman numerals: i), ii), iii), iv), etc are to denote a passage of time.

Unless specifically stated, the figures are not drawn to scale.

FIG. 1(A) demonstrates an embodiment of generating a linear physical map along the length of a long nucleic acid molecule by cleaving the molecule at known recognition sites producing an ordered pattern of lengths.

FIG. 1(B) demonstrates an embodiment of generating a linear physical map by attaching label bodies at known recognition sites producing an ordered pattern of segments.

FIG. 1(C) demonstrates an embodiment of generating a linear physical map by attaching label bodies along the length of molecule in a manner such the density of the labeling bodies correlates with the underlying AT/CG ratio

FIG. 2 demonstrates different, non-limiting embodiments of confined and non-confined channel types within a fluidic device.

FIG. 3(A) demonstrates an example constriction device capable of interrogating a long nucleic acid molecule, in which the signal generated during interrogation is the modulation of the blockade current through the constriction region as the macromolecule translocates said region. In some embodiments the long nucleic acid molecule has at least one labelling body bound to it during translocation. In some embodiments, the long nucleic acid molecule has no labelling bodies bound to it during translocation.

FIG. 3(B) demonstrates an example constriction device capable of interrogating a long nucleic acid molecule, in which the signal generated during interrogation is the modulation of the current between an electrode gap within the constriction region as the macromolecule translocates said region. In some embodiments the long nucleic acid molecule has at least one labelling body bound to it during translocation. In some embodiments, the long nucleic acid molecule has no labelling bodies bound to it during translocation.

FIG. 3(C) demonstrates an example constriction device capable of interrogating a long nucleic acid molecule, in which the signal generated during interrogation is the modulation of a transistor current from source to drain as the macromolecule translocates the constriction region of the device. In some embodiments the long nucleic acid molecule has at least one labelling body bound to it during translocation. In some embodiments, the long nucleic acid molecule has no labelling bodies bound to it during translocation.

FIG. 3(D) demonstrates an example constriction device capable of interrogating a long nucleic acid molecule, in which the signal generated during interrogation is the modulation of the current into an electrode within the constriction region as the macromolecule translocates said region. In some embodiments the long nucleic acid molecule has at least one labelling body bound to it during translocation. In some embodiments, the long nucleic acid molecule has no labelling bodies bound to it during translocation.

FIG. 4(A) demonstrates an example of a long nucleic acid molecule with AT/CG density labelling bodies translocating through a current blockade constriction device.

FIG. 4(B) demonstrates an example current trace generated by the device shown in FIG. 4(A).

FIG. 4(C) demonstrates an example of binned feature density profile generated from the current trace shown in FIG. 4(B).

FIG. 5 demonstrates various embodiments of AT/CG density linear physical maps.

FIG. 6 demonstrates an example of a long nucleic acid molecule in a partially melted state translocation through a current blockade constriction device.

FIG. 7 demonstrates (i) a long nucleic acid molecule with a higher order structure comprising of a loop approaching a current blockade constriction device, and (ii) said molecule translocating said device.

FIG. 8(A) demonstrates a long nucleic acid molecule with a higher order structure comprising of histones translocating through a current blockade constriction device.

FIG. 8(B) demonstrates a long nucleic acid molecule with a higher order structure comprising of TADs translocating through a current blockade constriction device.

FIG. 9 demonstrates (i) a long nucleic acid molecule with a higher order structure unable to translocate through a constriction device, and (ii) said molecule able to translocate said device after being exposed to enzymes that remove said higher order structure.

FIG. 10 demonstrates a multi-constriction device in which (i) a long nucleic acid molecule with a higher order structure is successfully translocating through the first of two constrictions in said device, and (ii) said long nucleic acid molecule unable to successfully translocate through the second of two constrictions in said device.

FIG. 11 demonstrates multi-constriction device in which a long nucleic acid molecule can be interrogated by any from a selection of constrictions that comprises the device, in which the constrictions are all of a different size.

FIG. 12(A) demonstrates a current blockade constriction device with retarding and collection fluidic channels for the long nucleic acid molecule.

FIG. 12(B) demonstrates a current blockade constriction device with a retarding region.

FIG. 13(A) demonstrates a method for generating a retarding force on a long nucleic acid molecule translocating through a constriction region by interaction of said molecule with a porous material, shown here as patterned fluidic features, wherein the fluidic features apply a frictional force on said molecule.

FIG. 13(B) demonstrates a method for generating a retarding force on a long nucleic acid molecule translocating through a constriction region by attachment of said molecule to a body.

FIG. 13(C) demonstrates a method for generating a retarding force on a long nucleic acid molecule translocating through a constriction region by interaction of said molecule with a fluid flow that applies a shear force on said molecule.

FIG. 13(D) demonstrates a method for generating a retarding force on a long nucleic acid molecule translocating through a constriction region by interaction of said molecule with an entropic barrier.

FIG. 14(A) demonstrates a method for retarding a long nucleic acid molecule translocation through a constriction region with a frictional force applied to a portion of the molecule by fluidic features.

FIG. 14(B) demonstrates a method for retarding a long nucleic acid molecule translocation through a constriction region with a fluid flow that applies a force on said molecule, directing said molecule against a porous material, thus generating a fictional force between said molecule and porous material.

FIG. 14(C) demonstrates a method for retarding a long nucleic acid molecule translocation through a constriction region with a fluid flow that generates a shear force on said molecule.

FIG. 15 demonstrates a method for interrogating a long nucleic acid molecule with constriction device that comprises a constriction region with a size transition from the opening of said region to the critical dimension of said region, such that physical conformation of the structure within said region changes as the molecule is translocated i) from the wider entrance of the region, ii) to the narrower critical dimension.

### DETAILED DESCRIPTIONS

### Definitions

As used herein, “about” or “approximately” in the context of a number shall refer to a range spanning+/−10% of the number, or in the context of a range shall refer to an extended range spanning from 10% below the lower limit of the listed range to 10% above the listed upper limit of the range.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The use of the term “combination” is used to mean a selection of items from a collection, such that the order of selection does not matter, and the selection of a null set (none), is also a valid selection when explicitly stated. For example, the unique combinations including the null of the set {A,B} that can be selected are: null, A, B, A and B.

Nucleic Acid. The terms “nucleic acid”, “nucleic acid molecule”, “oligonucleotide” and “polynucleotide”, “nucleic acid polymer”, “nucleic acid fragment”, “polymer” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNAs (mRNA), transfer RNAs, ribosomal RNAs, lncRNAs (Long noncoding RNAs), lincRNAs (long intergenic noncoding RNAs), ribozymes, cDNA, ecDNAs (extrachromosomal DNAs), artificial minichromosomes, cfDNAs (circulating free DNAs), ctDNAs (circulating tumor DNAs), cffDNAs (cell free fetal DNAs), recombinant polynucleotides, branched polynucleotides, plasmids, vectors, isolated DNA of any sequence, control regions, isolated RNA of any sequence, nucleic acid probes, and primers.

Unless specifically stated otherwise, the nucleic acid molecule can be single stranded, double stranded, or a mixture there-of. For example, there may be hairpin turns or loops.

Long Nucleic Acid Molecule. Unless specifically stated otherwise, a “long nucleic acid fragment” or “long nucleic acid molecule” is double strand nucleic acid of at least 1 kbp in length, and is thus a kind of macromolecule, and can span to an entire chromosome. It can originate from any source, man-made or natural, including single cell, a population of cells, droplets, an amplification process, etc. It can include nucleic acids that have additional structure such as structural proteins histones, and thus includes chromatin. It can include nucleic acid that has additional bodies bound to it, for example labeling bodies, DNA binding proteins, RNA.

Higher Order Nucleic Acid Structure. A “higher order nucleic acid structure”, or simply “structure” refers to any 2nd, 3rd, or 4th order DNA structure, including anybody bound to said nucleic acid molecule. The nucleic acid molecule may be linear or circular. Nucleic acids can have any of a variety of structural configurations, e.g., be single stranded, double stranded, triplex, replication loop or a combination of both, as well as having higher order intra- or inter-molecular secondary/tertiary/quaternary structures, e.g., chromosomal territories, compartments, Topologically Associating Domains (TAD), chromatin loop and local direct regulatory factors binding, condensing associated loops, cohesin associated loops, guide nucleic acid, argonaut complexes, CRISPR Cas9 complexes, nucleoprotein complexes, insulator complexes, enhancer-promoter complexes, ribonucleic acid (RNA), small interfering RNA (siRNA), micro RNA (miRNA), guide RNA (gRNA), long non-coding RNA (lncRNA), repeat region binding proteins, telomere modification proteins, nucleic acid repair proteins, regulatory factor binding proteins, nucleic acid binding proteins, proteins, histone deacetylase (HDAC), chromatin remodeling protein, methyl-binding protein, transcription factor transcription complexes, bending with kinks of the genomic DNA polymers such as hairpins, replication loops, triple stranded regions, in cis or trans fashion etc. The nucleotides within the nucleic acid may have any combination of epigenomic state including but not limited to such as methylation or acetylation states. The nucleic acid can originate from any source, man-made or natural, including single cell, a population of cells, droplets, an amplification process, etc. In some embodiments, these structures include compounds and/or interactions of nucleic acids and proteins. In some embodiments, these structures include 2D and 3D configurations of the nucleic acid beyond the linear 1D polymer chain. These 2D and 3D configurations can be formed via interactions with proteins, other nucleic acid molecules, or external boundary conditions. Non limiting examples of boundary conditions include a micro or nanofluidic chamber, a well on or in substrate or defined within a fluidic device, a droplet, a nucleus. The nucleic acid can include nucleic acids that has additional structure such as structural proteins including but not limited to such as any regulatory binding sites complexes, enhancer/transcription factor complex and their interaction with a nucleic acid molecule, Cohesins, condesins, CTCF proteins, PDS5 proteins, WAPL proteins, SA1, SA2, condensin I, condensin II, histones and their derivative complexes, and thus includes chromatin.

In particular, higher order nucleic acid structure can refer to the various levels of genome organization contained within a cell nucleus [Jerkovic, 2021], [Kempfer, 2020] either individually, collectively, or a sub-set there-of Such genomic organization starts with DNA winding around histones to form nucleosomes, which are organized into clutches, each containing ˜1-2 kb of DNA. Nucleosome clutches form chromatin nanodomains (CNDs) ˜100 kb in size, where most enhancer-promoter (E-P) contacts take place. At the scale of ˜1 Mb, CNDs and CCCTC-binding factor (CTCF)—cohesin-dependent chromatin loops form topologically associating domains (TADs) and loop domains. On the higher scale up to 100s of megabases, chromatin segregates into gene-active and gene-inactive compartments (A and B, respectively) and into compartment-specific contact hubs. At the highest topological level, the nucleus is organized into chromosome territories.

Hybridization. As used herein, the terms “hybridization”, “hybridizing,” “hybridize,” “annealing,” and “anneal” are used interchangeably in reference to the pairing of complementary or substantially complementary nucleic acids. Hybridization and the strength of hybridization (i.e., the strength of the association between the nucleic acids) is influenced by such factors as the degree of complementary between the nucleic acids, stringency of the conditions involved, the Tm (melting temperature) of the formed hybrid, and environmental conditions such as temperature and pH. “Hybridization” methods involve the annealing of one nucleic acid to another, complementary nucleic acid, i.e., a nucleic acid having a complementary nucleotide sequence.

Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In the context of this document, where hybridization occurs between nucleic acid strand and a double-stranded nucleic acid molecule, it should be understood that such hybridization is being done under conditions of either partial or full denaturation of the double-stranded nucleic acid molecule, unless otherwise specifically stated.

Labelling Body. A “labelling body” used herein is a physical body that can bind to a nucleic acid molecule, or to a body directly or indirectly bound to a nucleic acid molecule, which can be used to generate a signal that can be detected with interrogation, that differs from a detected signal (or lack there-of) that would be generated by said nucleic acid without said body. A labelling body may be a fluorescent intercalating dye that when bound to nucleic acid, can be used in a fluorescent imaging system to identify the presence of said nucleic acid. In another example, a labelling body may by a compound that binds specifically to methylated nucleotides, and gives a current blockade signal when transported through a nanopore, thus reporting a signal as to said molecule's methylation state. In another example, a fluorescent probe specifically hybridized to a sequence of a nucleic acid, thus providing confirmation with a fluorescent imaging system that the sequence is present on said nucleic acid. In another example, a fluorescent probe specifically binds to a specific protein (eg: DNA binding protein), with said protein bound to a long nucleic acid molecule. In some cases, the absence of the labelling body, is itself the signal. In some cases, the signal associated with the labeling body is an attenuation, blocking, displacement, quenching, or modification of a signal from another labeling body. Non-limiting examples include: binding of a dark labeling body to the nucleic acid to displace an existing bond fluorescent body; binding of a dark labeling body to the nucleic acid to block a fluorescent labeling body from binding; quenching a near-by fluorescent labeling body bond to a nucleic acid; directly, or indirectly, reacting with a fluorescent labeling body bond to a nucleic acid to reduce its fluorescence. In some cases, the labelling body is not physically attached to the nucleic molecule at the time of interrogating said nucleic molecule and labelling body. For example, a labelling body may be attached to a nucleic acid molecule via a cleavable linker. At the desired time, the linker is cleaved, releasing said labelling molecule which is then detected by interrogation.

Interrogation. “Interrogation” is a process of assessing the state of a nucleic acid. In some embodiments, the state of nucleic acid is assessed by assessing the state of at least one labeling body on the nucleic acid by measuring a signal generated directly, or indirectly from the at least one labeling body. It may be a binary assessment, such as the labeling body is present, or not. It may be quantitative such as how many labeling bodies are present on a molecule. It may be a trace of the density and/or physical count of labeling bodies along the length the molecule in relation to the molecule's physical structure. The signal may be fluorescent, electrical, magnetic, physical, chemical. The signal may be analog or digital in nature. For example, the signal may be an analog density profile of the labeling body along the length of the nucleic acid. In some embodiments, the state of the nucleic acid is directly interrogated without a labelling body. Non exhaustive examples of different interrogation methods include fluorescent imaging, bright-field imaging, dark-field imaging, phase contrast imaging, super resolution imaging, current, voltage, power, capacitive, inductive, or reactive measurement, nanopore sensing (both column blockade through the pore, and tunneling across the pore), chemical sensing (eg: via a reaction), physical sensing (eg: interaction with a sensing probe), SEM, TEM, STM, SPM, AFM. In addition, combinations of different labeling bodies and interrogation methods are also possible. For example: fluorescent imaging of an intercalating dye on a nucleic acid, while translocating said nucleic acid through a nanopore and measuring the pore current.

Sequence. The term “sequence” or “nucleic acid sequence” or “oligonucleotide sequence” refers to a contiguous string of nucleotide bases and in particular contexts also refers to the particular placement of nucleotide bases in relation to each other as they appear in an oligonucleotide.

Sequencing can be performed by various systems currently available, such as, with limitation, a sequencing system by Illumina, Pacific Biosciences, Oxford Nanopore, Life Technologies (Ion Torrent), BGI.

Phasing. “Phasing” is the task or process of assigning genetic content to either the paternal or material chromosomes. The genetic content can be a nucleic acid molecule, a sequence, or a consensus from a set of sequences. The genetic content can be a single nucleic acid molecule whose sequence content may be known, unknown, or partially known. For example, it may be determined that a nucleic acid molecule originates from the mother, however the sequence content of said molecule is completely, or partially, unknown.

In some embodiments, within the context of this disclosure, phasing also refers to the identification that two separate genetic contents originate from the same maternal or paternal chromosome, however it may not be known to which; or that the two separate genetic contents originate from a different chromosome (one to the maternal, the other to the paternal), however again it may not be known to which. The said genomic content, in the concept of “genomic phasing”, could be further expanded from separating the primary linear nucleic acid sequence information in the context of paternal, maternal, chromosomal, sister chromatids and extra-chromosomal entities, to include its native epigenomic information associated with the sequence, and to include the next level of secondary/tertiary/quaternary structures associated with the underlying sequence information, on maternal, paternal, chromosomal, sister-chromatids, large genomic regions and include but not limited to extra-chromosomal genomic entities, that were naturally occurring such as ecDNA or man-made artificial mini-chromosomes.

Structural Variation. As used herein, “structural variation”, “structural variant”, or “SV” is the variation in structure of an organism's chromosome with respect to a genomic reference. These variations include a wide variety of different variant events, including insertions, deletions, duplications, retrotransposition, translocations, inversions short and long tandem repeats, rearrangements, and the like. These structural variations are of significant scientific interest, as they are believed to be associated with a range of diverse genetic diseases. In general, the operational range of structural variants includes events >50 bp, while the “large structural variations” typically denotes events >1,000 bp or more. The definition of structural variation does not imply anything about frequency or phenotypical effects.

Reference. A “genomic reference” or “reference” is any genomic data set that can be compared to another genomic data set. Any data formats may be employed, including but not limited to sequence data, karyotyping data, methylation data, genomic functional element data such as cis-regulatory element (CRE) map, primary level structural variant map data, higher order nucleic acid structure data, physical mapping data, genetic mapping data, optical mapping data, raw data, processed data, simulated data, signal profiles including those generated electronically or fluorescently. A genomic reference may include multiple data formats. A genomic reference may represent a consensus from multiple data sets, which may or may not originate from different data formats. The genomic reference may comprise a totality of genomic information of an organism or model, or a subset, or a representation. The genomic reference may be an incomplete representation of the genomic information it is representing.

The genomic reference may be derived from a genome that is indicative of an absence of a disease or disorder state or that is indicative of a disease or disorder state. Moreover, the genomic reference (e.g., having lengths of longer than 100 bp, longer than 1 kb, longer than 100 kb, longer than 10 Mb, longer than 1000 Mb) may be characterized in one or more respects, with non-limiting examples that include determining the presence (or absence) of a particular feature, a particular haplotype, a particular genetic variations, a particular structural variation, a particular single nucleotide polymorphism (SNP), and combinations thereof, referring not only to being present or absent from the genomic reference in its entirety, but also from a particular region of genomic reference, as defined by the neighboring genomic content. Moreover, any suitable type and number of characteristics of the genomic reference can be used to characterize the sample nucleic acid, as derived (or not derived) from a nucleic acid indicative of the disorder or disease based upon whether or not it displays a similar character to the reference.

In some cases, the genomic reference is a physical map. This can be generated in any number of ways, including but not limited to: raw single molecule data, processed single molecule data, an in-silico representation of a physical map generated from a sequence or simulation, an in-silico representation of a physical map generated by assembling and/or averaging multiple single molecule physical maps, or combination there-of. For example, based on a known, or partially known sequence, a simulated in-silico physical map can be generated based on the method of generating a physical map used. In an embodiment where-by the physical map comprises labelling bodies at known sequences, a discrete ordered set of segment lengths in base-pairs can be generated. In an embodiment where-by the physical map comprises a continuous analog signal of labeling signal density along the sequence length, in base-pairs based on simulated local hydrogen bonds dissociation kinetics between the double helices, in chemical moiety modification, regulatory factor association or structural folding patterns based on nucleotide sequence and predicted functional element database maps.

In some cases, the genomic reference is data obtained from microarrays (for example: DNA microarrays, MMChips, Protein microarrays, Peptide microarrays, Tissue microarrays, etc), or karyotypes, or FISH analysis. In some cases, the genomic reference is data obtained from indirect 3D Mapping technologies.

In some cases, characterizations of the comparison with the genomic reference may be completed with the aid of a programmed computer processor. In some cases, such a programmed computer processor can be included in a computer control system.

Physical Mapping. “Physical mapping” or “mapping” of nucleic acid comprises a variety of methods of extracting genomic, epigenomic, functional, or structural information from a physical fragment of long nucleic acid molecule, in which the information extracted can be associated with a physical coordinate on the molecule. As a general rule, the information obtained is of a lower resolution than the actual underlying sequence information, but the two types of information are correlated (or anti-correlated) spatially within the molecule, and as such, the former often provides a ‘map’ for sequence content with respect to physical location along the nucleic acid. In some embodiments, the relationship between the map and the underlying sequence is direct, for example the map represents a density of AG content along the length of the molecule, or a frequency of a specific recognition sequence. In some embodiments, the relationship between the map the underlying sequence is indirect, for example the map represents the density of nucleic acid packed into structures with proteins, which in turn is at least partially a function of the underlying sequence. In some embodiments, the physical map is a linear physical map, in which the information extracted can be assigned along the length of an axis, for example, the AT/CG ratio along the major axis of long nucleic acid molecule. In the preferred embodiment, the linear (or 1D) physical map is generated by interrogating labeling bodies that are bound along an elongated portion of a long nucleic acid molecule's major axis. For clarity, a string occupying 3D space in a coiled state can be represented as straight line, and thus extracted values along the 3D coil, can be represented as binned values along a 1D representation of the string, and thus constitute a linear physical map. In some embodiments, the physical map is a 2D physical map, in which the information extracted can be assigned within a plane that comprises the molecule, for example: karyotyping. In some embodiments, the physical map is a 3D physical map, in which the information extracted can be assigned in 3D volume in which the molecule occupies. For example, tagging with super-resolution techniques to identify in (x,y,z) space the location of the tag within the chromosome as demonstrated with OligoFISSEQ [Nguyen, 2020], or in-situ genome sequencing [Payne, 2020].

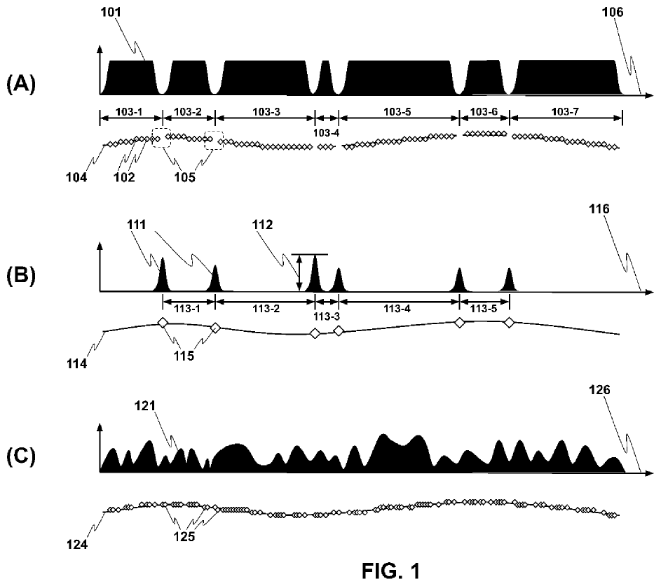

The first and most widely used form of physical mapping is karyotyping, where-by metaphase chromosomes are treated with a stain process that preferentially binds to AT or CG regions, thus producing ‘bands’ that correlate with the underlying sequence as well as the structural and epigenomic patterns of the nucleic acid [Moore, 2001]. However, the resolution of such a process with respect to nucleotide sequence is quite poor, about 5-10 Mbp, due to the condensed nature of nucleic acid being imaged. More recent methods of using linear mapping of elongated interphase genomic DNA have been generated by imaging nucleic acid digested at known restriction sites [Schwartz, 1988, U.S. Pat. No. 6,147,198] (eg: see FIG. 1(A)), imaging attached fluorescent probes at nicking sites [Xiao, 2007] (eg: see FIG. 1(B)), imaging the fluorescent signature of a nucleic acid molecule's methylation pattern [Sharim, 2019], imaging the fluorescent signature of a chromatin's histone [Riehn, 2011], electrical detection of bound probes to a nucleic acid through a sensor [Rose, 2013, 2014/0272954], and electrical detection of the methylation signature on a nucleic acid using a nanopore sensor [Rand, 2017].

Another method of linear physical mapping is to measure the AT/CG relative density or local melting temperature along the length of an elongated nucleic molecule (eg: see FIG. 1(C)). Such a signal can either be used to compare against other similar maps, or against a map generated in-silico from sequence data. There are many ways of generating such a signal. For example, the signal can be fluorescent or electrical in nature. Nucleic acid can be uniformly stained with an intercalating dye, and then partially melted resulting in the relative loss of dye in regions of rich AT content [Tegenfeldt, 2009, U.S. Pat. No. 10,434,512]. Another method is to expose double stranded nucleic acid to two different species that compete to bind to the nucleic acid. One species is non-fluorescent and preferentially binds to AT rich regions, while the other species is fluorescent and has no such bias [Nilsson, 2014]. Yet another method is to use two different color dyes that differentially label the AT and CG regions.

Mapping using such non-condensed interphase nucleic acid polymer strands has improved upon the resolution of the primary sequence information, however the maps were stripped of any native structural folding or bound supporting proteins information and are often extracted from bulk solution of pooled samples with many potentially heterogeneous cells. Recently, 3D physical maps have been demonstrated where-by fluorescent tags attached to chromosomes as specific locations are interrogated to determine their relative position within the chromosome in 3D space. See [Kempfer, 2020] for a review of the various methods.

FIG. 1 demonstrates a variety of different embodiments for generating and interrogating a long nucleic acid molecule linear physical map. In FIG. 1(A), a physical map of a long nucleic acid molecule 104 is generated by cleaving the molecule at particular sequence sites (eg: recognition sites for restriction enzymes) thus resulting in gaps 105 where the cleaving event took place. Along the length of a molecule, a dye is attached non-specifically (eg: using an intercalating dye) such that child molecules from the originating the parent molecule can be interrogated to generate a signal 101 that follows the physical length (0106) of the parent molecule. The signal can then be used determined the lengths and order of the individual child molecules {103-x}, and thus generating the parent molecule's physical map. In most embodiments of this method, the parent molecule is combed onto a surface and then cleaved, so as to maintain physical proximity and relative order of the child molecules. However, such an embodiment could also be implemented in at least a partially elongated state within an elongating channel of a confined fluidic device such that the order of the child molecules can be interrogated [Ramsey, 2015, U.S. Pat. No. 10,106,848]. In some embodiments, a mixture of different cleaving sites may be used simultaneously.

In FIG. 1(B), a physical map of a long nucleic acid molecule 114 is generated by sparsely binding label bodies 115 along the length of the molecule, with the binding sites correlated (or anti-correlated) with a set of specific target(s). In some methods, the labeling body is bound directly to a sequence motif target. In some methods, the labeling body generating a signal is bound indirectly via a process, for example: a sequence specific nick is generated, followed by incorporation of nucleotides starting at the nick site, some of which may be capable of generating a signal. The long nucleic acid molecule with labeling bodies is interrogated, generating signals 111 from the label bodies 115 along the physical length of the molecule 116. The distance between the signals, a collection of lengths and orders {113-x} then represents the molecule's physical map. In some embodiments, further information can be generated by also interpreting the relative magnitudes of the signals 112 from the various labeling sites. When fluorescent interrogation is used, different color labeling bodies can be used to represent different specific sites.

In FIG. 1(C), a physical map of a long nucleic acid molecule 124 is generated by densely binding labeling bodies 125 along the length of the molecule, such that the binding pattern correlates (or anti-correlates) with the underlying physical sequence content of the molecule. For example, the relative AT/CG content, or the relative melting temperature, or the relative density of methylated CGs. Due to the dense nature of the labeling bodies in this method, the physical map is not a collection of lengths and orders, but rather an analog signal 121 that varies in intensity along the physical length of the molecule 126.

The method of interrogation to generate a physical map is typically fluorescent imaging, however different embodiments are also possible, including a scanning probe along the length of a combed molecule on a surface, or a constriction device that measures the coulomb blockade current through or tunneling current across the constriction as the molecule translocate through.

Unless specifically stated otherwise, a physical map refers to any of the previously mentioned methods, including combinations there-of. For example, a long nucleic acid molecule may have a physical map generated from the AT/TC density with a fluorescent labelling body along the length of the molecule, and then also have a physical map generated from the methylation profile along the length of the molecule by constriction device as the molecule is transported through said constriction device.

Elongated Nucleic Acid. The majority of linear physical mapping methods that use fluorescent imaging or electronic signals to extract a signal related to the underlying genomic, structural, or epigenomic content employ some form of method to at least locally ‘elongate’ the long nucleic acid molecule such that the resolution of the physical mapping in the region of elongation can be improved, and disambiguates reduced. A long nucleic acid molecule in its natural state in a solution will form a random coil. Thus, a variety of methods have been developed to ‘uncoil’ and elongate the molecule.

By binding a portion of long nucleic acid molecules on a functionalized solid surface, the molecule is elongated by flowing a solution and ultimately pulled taut, coming into full contact with the substrate surface [Bensimon, 1997, U.S. Pat. No. 7,368,234], a technique typically called ‘combing’ DNA. Alternatively, there are other long polymer elongation methods such as fluid flow induced elongation with ends anchoring on surface [Gibb, 2012], aqueous solution hydrodynamic focusing by laminar flows [Chan, 1999, U.S. Pat. No. 6,696,022], linearization by confining nanochannels [Tegenfeldt, 2005], long nucleic acid molecules in microfluidic device pulled by two angled opposing externally applied forces in a presence of physical obstacle features[Volkmuth, 1992], molecules hydrodynamically trapped in a fluidic device by simultaneously exposed to two opposing externally applied forces [Tanyeri, 2011].

Most of the time, the elongation state of at least a portion of the long nucleic acid molecule has to be sustained by an external force before otherwise returning to its natural random coiled state, unless at least a portion of the nucleic acid is retained in the elongated state by physical confinement without a sustaining external force [Dai, 2016].

Unless specifically stated otherwise, an ‘elongated’ or ‘partially elongated’ nucleic acid is a long nucleic acid fragment for which at least one segment of the major axis of the molecule comprising at least 1 kb can be projected against a 2D plane, and does not overlap with itself. For clarity, for embodiments where-by long nucleic acid includes additional structure, for example as when the nucleic acid is contained in chromatin, compacted with histones, the major axis refers to the larger chromatin molecule, not the nucleic acid strand itself. Therefore statements in this disclosure such as “along the length of the molecule” when referring to long nucleic acid molecules, refers to along the length of the major axis.

Indirect 3D Mapping. In this document, “indirect 3D mapping” refers to protocols that involve capturing the proximity relationship of at least two strands of nucleic acid, either of the same chromosome or not. For reference [Kempfer, 2020], and [Szabo, 2019] reviews these various techniques, of which a non-exhaustive list includes the following: 3C, 4C, 5C, Hi-C, TCC, PLAC-seq, ChIA-PET, Capture-C, C-HiC, Single-Cell HiC, GAM, SPRITE, ChIA-Drop.

Binding. “Binding”, “bound”, “bind” as used herein generally refers to a covalent or non-covalent interaction between two entities (referred to herein as “binding partners”, e.g., a substrate and an enzyme or an antibody and an epitope). Any chemical binding between two or more bodies is a bond, including but not limited to: covalent bonding, sigma bonding, pi ponding, ionic bonding, dipolar bonding, metalic bonding, intermolecular bonding, hydrogen bonding, Van der Waals bonding. As “binding” is a general term, the following are all examples of types of binding: “hybridization”, hydrogen-binding, minor-groove-binding, major-groove-binding, click-binding, affinity-binding, specific and non-specific binding. Other example include: Transcription-factor binding to nucleic acid, protein binding to nucleic acid.

Specifically Binds. As used herein, the terms “specifically binds” and “non-specifically binds” must be interpreted in the context for which these terms are used in the text. For example, a body may “specifically bind” to a nucleic acid molecule but have no significant preference or bias with respect the underlying sequence of said nucleic acid molecule over some genomic length scale and/or within some genomic region. As such, in the context of molecule's sequence, the body “non-specifically binds” to said nucleic acid molecule.

When in the context of binding between physically distinct molecules, “Specific binding” typically refers to interaction between two binding partners such that the binding partners bind to one another, but do not bind other molecules that may be present in the environment (e.g., in a biological sample, in tissue) at a significant or substantial level under a given set of conditions (e.g., physiological conditions).

Preferentially Binds. The term “preferentially binds” means that in comparison between at least two different binding sites (the sites can be on the same entity, or can be physically different entities), there is a non-zero probability of binding between a certain body and both sites, however conditions can exist in which the probability of binding of the certain body is preferable at one site over another.

Microfluidic Device. The term “microfluidic device” or “fluidic device” as used herein generally refers to a device configured for fluid transport and/or transport of bodies through a fluid, and having a fluidic channel in which fluid can flow with at least one minimum dimension of no greater than about 100 microns. The minimum dimension can be any of length, width, height, radius, or cross-sectional axis. A microfluidic device can also include a plurality of fluidic channels. The dimension(s) of a given fluidic channel of a microfluidic device may vary depending, for example, on the particular configuration of the channel and/or channels and other features also included in the device.

Microfluidic devices described herein can also include any additional components that can, for example, aid in regulating fluid flow, such as a fluid flow regulator (e.g., a pump, a source of pressure, etc.), features that aid in preventing clogging of fluidic channels (e.g., funnel features in channels; reservoirs positioned between channels, reservoirs that provide fluids to fluidic channels, etc.) and/or removing debris from fluid streams, such as, for example, filters. Moreover, microfluidic devices may be configured as a fluidic chip that includes one or more reservoirs that supply fluids to an arrangement of microfluidic channels and also includes one or more reservoirs that receive fluids that have passed through the microfluidic device. In addition, microfluidic devices may be constructed of any suitable material(s), including polymer species and glass, or channels and cavities formed by multi-phase immiscible medium encapsulation. Microfluidic devices can contain a number of microchannels, valves, pumps, reactor, mixers and other components for producing the droplets. Microfluidic devices may contain active and/or passive sensors, electronic and/or magnetic devices, integrated optics, or functionalized surfaces. The physical substrates that define the microfluidic device channels can be solid or flexible, permeable or impermeable, or combinations there-of that can change with location and/or time. Microfluidic devices may be composed of materials that are at least partially transparent to at least one wavelength of light, and/or at least partially opaque to at least one wavelength of light.

A microfluidic device can be fully independent with all the necessary functionality to operate on the desired sample contained within. The operation may be completely passive, such as with the use of capillary pressure to manipulate fluid flows [Juncker, 2002], or may contain an internally power supply such as a battery. Alternatively, the fluidic device may operate with the assistance of an external device that can provide any combination of power, voltage, electrical current, magnetic field, pressure, vacuum, light, heat, cooling, sensing, imaging, digital communications, encapsulation, environmental conditions, etc. The external device maybe a mobile device such as a smart phone, or a larger desk-top device.

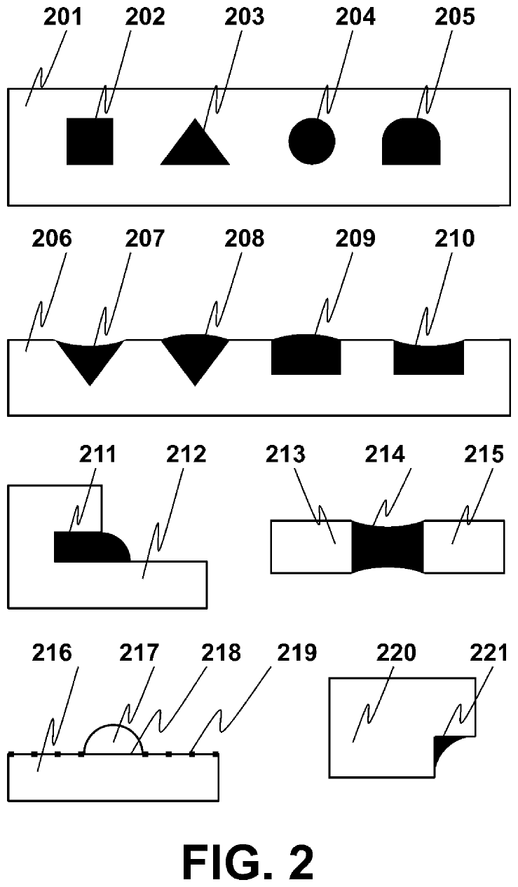

The containment of the fluid within a channel can be by any means in which the fluid can be maintained within or on features defined within or on the fluidic device for a period of time. In most embodiments, the fluid is contained by the solid or semi-solid physical boundaries of the channel walls. FIG. 2 shows an example where-by channel walls with cross-sections such as rectangles (202), triangles (203), ovals (204), and mixed geometry (205) are all defined within a fluidic device (201). In other embodiments, fluidic containment within the fluidic device may be at least partially contained via solid physical features in combination with surface energy features [Casavant, 2013], or an immiscible fluid [Li, 2020]. Examples of a fluid being at least partially confined within physical boundaries include various channels physically defined on the surface of a fluidic device (206) such as grooves (207, 208) and rectangles (209, 210), all of which are filled with liquid of sufficiently minimal quantity, that surface tension allows for the liquid to be physically maintained within the channels, and not overflow. In other embodiments, the channel (211) could be a defined by a groove in a corner (212) of a fluidic device, or the channel (214) could be defined by two physically separated boundaries (213 and 215) of a fluidic device, or the channel (221) could be defined by a corner (220) of a fluidic device. In other embodiments, the channel (217) is defined by a hydrophilic section (218) on the surface of a fluidic device (316) where-by the hydrophilic section is bounded by hydrophobic sections (219) on the surface of the fluidic device. In all cases, these embodiments are non-limiting examples.

In some embodiments, the fluidic device includes an “electrowetting device” or “droplet microactuator”, which is a type of microfluidic device capable of controlled droplet operations within the fluidic device via specific application of local electric fields. Non limiting examples of such devices include a liquid droplet surrounded by air on an open surface, and a liquid droplet surrounded by oil sandwiched between two surfaces. A detailed review of the various configurations of use, and physics of droplet control are provided by [Mugele, 2005] and [Zhao, 2013], both of which are provided here for reference.

It should be understood that some of the principles and design features described herein can be scaled to larger devices and systems including devices and systems employing channels and features reaching the millimeter or even centimeter scale channel cross-sections. Thus, when describing some devices and systems as “microfluidic,” it is intended that the description apply equally, in certain embodiments, to some larger scale devices. In addition, it should be understood that some of the principles and design features described herein can be scaled to smaller devices and systems including devices and systems employing channels and features that are 100s of nanometers, or even 10s of nanometers, or even single nanometers in scale channel cross-sections. Thus, when describing some devices and systems as “microfluidic,” it is intended that the description apply equally, in certain embodiments, to some smaller scale devices. As an example, a device may have input wells to accommodate liquid loading from a pipette that are millimeters in diameter, which are in fluidic connection with channels that are centimeters in length, 100s of microns wide, and 100s of nm deep, which are then in fluidic connection with nanopore constriction devices that are 0.1-10 nm in diameter.

A variety of materials and methods, according to certain aspects of the invention, can be used to form articles or components such as those described herein, e.g., channels such as microfluidic channels, chambers, etc. For example, various articles or components can be formed from solid materials, in which the channels can be formed via micromachining, film deposition processes such as spin coating and chemical vapor deposition, laser fabrication, photolithographic techniques, bonding techniques, deposition techniques, lamination techniques, molding techniques, etching methods including wet chemical or plasma processes, multi-phase immiscible medium encapsulation and the like. For patterning, a variety of methods may be employed, including but not limited to: photolithography, electron-beam lithography, nanoimprint lithography, AFM lithography, STM lithography, focused ion-beam lithography, stamping, embossing, molding, and dip pen lithography. For bonding, a variety of methods may be employed, including but not limited to: thermal bonding, adhesive bonding, surface activated bonding, fusion bonding, anodic bonding, plasma activated bonding, laser bonding, and ultra sonic bonding.

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques [Xia, 1998, Whitesides, 2001].

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material. The device may be formed from glass, silicon, silicon nitride, silicon oxide, quartz. The device may be formed from a combination of different materials that are mixed, bonded, laminated, layered, joined, merged, or combination there-of.

Physical Obstacle. Unless specifically stated otherwise, a “physical obstacle” is a physical feature within a fluidic device in which a long nucleic acid molecule, in the presence of an applied force, physically interacts with, such that the molecule's physical conformation or location is different than had said physical obstacle not been present. Non-limiting examples include: pillars, corners, pits, traps, barriers, walls, bumps, constrictions, expansions. The physical obstacles need not be physically continuous with the fluidic channel, but may also be additive to the device, with non-limiting examples including: beads, gels, particles.

External Force. An “external force” is any applied force on a body such that the force that can perturb the body from a state of rest. Non-limiting examples include hydrodynamic drag exerted by a fluid flow [Larson, 1999] (which can be imitated by a pressure differential, gravity, capillary action, electro-osmotic), an electric field, electric-kinetic force, electrophoretic force, pulsed electrophoretic force, magnetic force, dielectric-force, centrifugal acceleration or combinations there-of. In addition, the external force may be applied indirectly, for example if bead is bound to the body, and then the bead is subjected to an external force such a magnetic field, or optical teasers.

Retarding Force. A “retarding force” is any force that retards a body's movement in the presence of an external force. Non-limiting examples include any of the following, or combination there-of: an entropic barrier, shear force, frictional force, Van der Waals force, a physical obstruction, binding to surface (such as a substrate or bead), a gel, an artificial gel. It should be noted that the retarding force need not keep the body motionless, or maintain a zero-average velocity. In some cases, the retarding force may itself be an external force, such that two external forces counter-act each other, one acting to retard the body's movement in the direction of the first external force.

Functionalize Surface. A “functionalized surface” is a surface that has been modified or engineered such as by certain chemicals, or macromolecules, to elicit certain desired properties. For example: to bind specifically or non-specifically to a macromolecule, or to provide a reagent.

Surface Energy. Surface tension of a fluid is the energy parallel to the surface that opposes extending the surface. Surface tension and surface energy are often used interchangeably. Surface energy is defined here as the energy required to wet a surface. To achieve optimum wicking, wetting and spreading, the surface tension of a fluid is decreased and is less than the surface energy, of the surface to be wetted. The wicking movement of a fluid through the channels of a fluid device occurs via capillary flow. Capillary flow depends on cohesion forces between liquid molecules and forces of adhesion between liquid and walls of channel. The Young/Laplace Equation states that fluids will rise in a channel or column until the pressure differential between the weight of the fluid and the forces pushing it through channel are equal. [Moore, 1962] Walter J. Moore, Physical Chemistry 3rd edition, Prentice-Hall, 1962, p. 730.

Δp=(2γ cos θ)/r

where Δp is the pressure differential across the surface, γ is the surface tension of the liquid, θ is the contact angle between the liquid and the walls of the channel and r is the radius of the cylinder. If the capillary rise is h and ρ is the density of the liquid then the weight of the liquid in the column is πr2ghρ or the force per unit area balancing the pressure difference is ghρ, therefore:

(2γ cos θ)/r=ghρ

For maximum flow through capillary channels, the radius of the channel should be small, the contact angle θ should be small and γ the surface tension of the fluid should be large. The theoretical explanation of this phenomenon can be described by the classical model know as Young's Equation:

γSV=γSL+γLV cos θ

which describes the relationship between the contact angle θ and surface tension of liquid-vapor interface γLV, the surface tension of the solid-vapor interface γSV, and surface tension of the liquid-vapor interface γSL. When the contact angle θ between liquid and solid is zero or so close to 0, the liquid will spread over the solid. A contact angle measurement test is used as an objective and simple method to measure the comparative surface tensions of solids. In general, a material is considered to be hydrophilic when the contact angle in this test is below 90°. If the contact angle is above 90°, the material is considered to be hydrophobic.

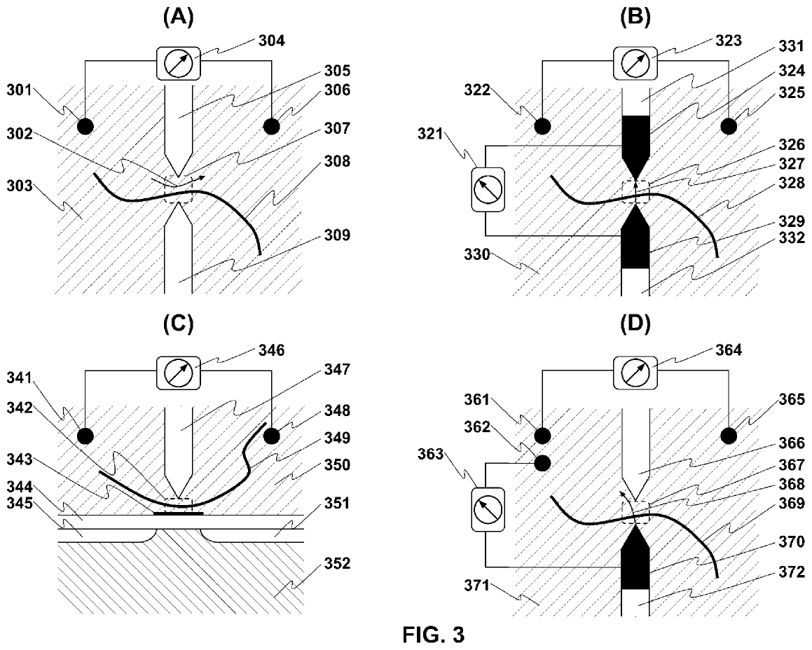

Constriction Device. The “constriction device” is a type of microfluidic device that consists of a small opening or threshold (a “constriction”, “pore”, “nanopore” or a “gap”) that fluidically connects two fluidic chambers through the constriction with a solution, from which an electrical signal can be modulated by macromolecules interacting with said constriction device, thus allowing for interrogation of said macromolecule by directly, or indirectly, monitoring the signal modulation. In all embodiments, the interaction involves at least one portion of said macromolecule being contained within said constriction. In some embodiments, the two fluidic chambers are only fluidically connected through the constriction. In some embodiments, there is at least one other fluidic connection that connects the two fluidic chambers. In some embodiments, the two fluidic chambers a single chamber of fluid. In some embodiments, the constriction is tangible. FIGS. 3(A), 3(B), 3(C), and 3(D) demonstrates 4 different constriction device embodiments with tangible constrictions, of which, a constriction device may be comprised of. In some embodiments, the constriction is intangible. For example, the constriction can be comprised of a force field that locally constricts the macromolecule as the macromolecule translocate through the constriction. The force field can be comprised of external force. In some embodiments, a constriction is comprised of fluid flow that results in a focusing of the flow into a constriction.

FIG. 3(A) shows an embodiment constriction device where-by the signal is the modulated current (302) through the constriction region (307) as the macromolecule (308) interacts with the constriction while being at least partially contained within said constriction. In this drawn embodiment, the currenting sourcing and sensing are performed by a source measurement unit (SMU) (304) via two electrodes (301, 306), each in electrical contact with the solution (303) that fluidically connects both sides of the constriction. Furthermore, said SMU also controls the macromolecule translocation. However, in other embodiments, the current sourcing, current sensing, and macromolecule translocation can all be performed by separate, or combination of devices, with separate, or combination of electrodes. In this drawn embodiment, the constriction region (307) opening is defined by surrounding material (305 and 309 which are physically connected).

FIG. 3(B) shows an embodiment constriction device where-by the signal is the modulated current (327) between two electrodes (324 and 329) that together form an electrode gap (which in this embodiment the constriction region (326) comprises said gap) as the macromolecule (366) is at least partially contained within with the constriction region. In some embodiments, the constriction region does not comprise the electrode gap, but rather, the electrode gap is in close proximity to the constriction region. In this drawn embodiment, the modulated current (327) is sourced and sensed by an SMU device 321 in electrical contact with the two electrodes, while the macromolecule translocation is controlled by a separate device (323) with electrical terminals (322 and 325) in electrical connection with the solution (330). In this drawn embodiment, the constriction region (326) opening is defined by a surrounding material (331 and 332 which are physically connected) which comprises the electrode gap.

FIG. 3(C) shows an embodiment constriction device where-by the signal is the modulated current between the source (345) and drain (351) of a semiconductor (352) transistor as the transistor gate (344) modulates the trans-conductivity of the transistor due to interaction of a sensing element (343) with a macromolecule (349) as said macromolecule is at least partially contained within the constriction region (342). In this drawn embodiment, the constriction region (342) comprises the sensing element (343). However, in other embodiments, the sensing element is in close proximity to the constriction region. In this draw embodiment, the macromolecule translocation is controlled by an electrical device (346) with electrode terminals (341 and 348) that are in electrical contact with the solution (350). In this drawn embodiment, the constriction region (342) opening is defined by a surrounding material (347) which comprises the sensor (343).

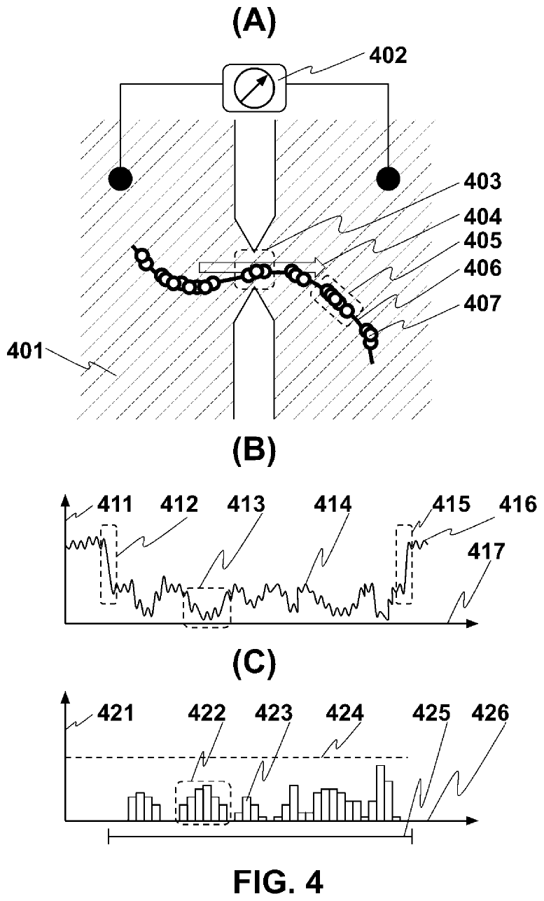

FIG. 4(D) shows an embodiment constriction device where-by the signal is the modulated current (368) between an electrode (370) within the constriction region (367) and a second electrode (362) in electrical contact with the solution (371) as the macromolecule (369) is at least partially contained within said region. In this drawn embodiment, the modulated current is sourced and sensed by an SMU (363), while the macromolecule translocation is controlled by an electrical device (364) with electrode terminals (361 and 365) that are in electrical contact with the solution (371). In some embodiments, the constriction region (367) does not comprise the current sensing electrode (370), but rather, said electrode is in close proximity to said constriction region. In this drawn embodiment, the constriction region (367) opening is defined by a surrounding material (366 and 372 which are physically connected) which comprises the electrode (370).

The constriction device opening can range from 1000 nm to 0.3 nm at its narrowest, and length along the long axis through which the nucleic acid translocates can range from 50,000 nm to 0.3 nm. The dimensions will be selected based on the application chosen, as the opening must be appropriately scaled to allow for a particular physical configuration of macromolecule to be interrogated.

The constriction device may consist of multiple constriction devices. In addition, a combination of all types of signal measurements are possible, either sharing the same constriction, or with physically different constrictions in fluidic connection with each other. Furthermore, multiple combinations of such constrictions in any serial and/or parallel combination that are in fluidic connection with each other are also possible.

The constriction can be composed of a biological material, a solid-state material, or a combination there-of.

The constriction device may be contained within a membrane, film, thin substrate, sheet, lipid bilayer or the like such that the constriction's major axis is normal to the surface, which itself may be largely composed of a biological or solid state material, or combination there-of Non-limiting examples include the following prior-art: [Akeson, 1995, patent], [Branton, 1999, Patent], [Deamer, 1999, patent]. The constriction device may be contained within a substrate such that its major axis is parallel to the surface. Non-limiting examples include the following: [Sohn, 1999, patent application] [Li, 1999, Patent] [Sauer, 2000, Patent] [Barth, 2003, patent].

A “constriction” specifically refers to a pore having an opening with a diameter at its most narrow point of about 0.3 nm to about 1000 nm. Pores useful in the present disclosure include any pore capable of permitting the linear translocation of a polymer or macro-molecule from one side to the other at a velocity amenable to monitoring techniques, such as techniques to detect current fluctuations. In some embodiments, the pore comprises a protein, such as alpha-hemolysin, Mycobacterium smegmatis porin A (MspA), OmpATb, homologs thereof, or other porins, as described in Gundlach, 2008, U.S. Pat. No. 8,673,550], [Gundlach, 2010, U.S. Pat. No. 9,588,079], [Gundlach, 2009, 2012/0055792], and [Manrao, 2012], each of which is incorporated herein by reference in its entirety. A “homolog,” as used herein, is a gene from another bacterial species that has a similar structure and evolutionary origin. By way of an example, homologs of wild-type MspA, such as MppA, PorM1, PorM2, and Mmcs4296, can serve as the. Protein pores have the advantage that, as biomolecules, they self-assemble and are essentially identical to one another. In addition, it is possible to genetically engineer protein pores to confer desired attributes, such as substituting amino acid residues for amino acids with different charges, or to create a fusion protein (e.g., an exonuclease+alpha-hemolysin). Thus, the protein pores can be wild-type or can be modified to contain at least one amino acid substitution, deletion, or addition.

In some embodiments, such as incorporating MspA protein pores, the pore comprises a vestibule and a constriction zone that together form a tunnel. A “vestibule” refers to the cone-shaped portion of the interior of the pore whose diameter generally decreases from one end to the other along a central axis, where the narrowest portion of the vestibule is connected to the constriction zone. A vestibule may generally be visualized as “goblet-shaped.” Because the vestibule is goblet-shaped, the diameter changes along the path of a central axis, where the diameter is larger at one end than the opposite end. The diameter may range from about 2 nm to about 1000 nm. When referring to “diameter” herein, one can determine a diameter by measuring center-to-center distances or atomic surface-to-surface distances.

In some embodiments, the pores can include or comprise DNA-based structures, such as generated by DNA origami techniques. For descriptions of DNA origami-based pores for analyte detection, see [Keyser, 2011, U.S. Pat. No. 10,330,639], incorporated herein by reference.

In some embodiments, the pore can be a solid state pore. Solid state pores can be produced as described in [Li, 1999, patent] and [Zhu, 2005, patent], incorporated herein by reference in their entireties. Solid state pores have the advantage that they are more robust and stable. Furthermore, solid state nanopores can in some cases be multiplexed and batch fabricated in an efficient and cost-effective manner. Finally, they might be combined with micro-electronic fabrication technology. In some embodiments, the pore comprises a hybrid protein/solid state pore in which a pore protein is incorporated into a solid state pore. In some embodiments, the pore is a biologically adapted solid-state pore.

In some cases, the pore is disposed within a membrane, thin film, or lipid bilayer, which can separate the first and second conductive liquid media, which provides a nonconductive barrier between the first conductive liquid medium and the second conductive liquid medium. The pore, thus, provides liquid communication between the first and second conductive liquid media. In some embodiments, the pore provides the only liquid communication between the first and second conductive liquid media. The liquid media typically comprises electrolytes or ions that can flow from the first conductive liquid medium to the second conductive liquid medium through the interior of the pore. Liquids employable in methods described herein are well-known in the art. Descriptions and examples of such media, including conductive liquid media, are provided in [Akeson, 1995, patent], for example, which is incorporated herein by reference in its entirety. The first and second liquid media may be the same or different, and either one or both may comprise one or more of a salt, a detergent, or a buffer. Indeed, any liquid media described herein may comprise one or more of a salt, a detergent, or a buffer. Additionally, any liquid medium described herein may comprise a viscosity-altering substance or a velocity-altering substance.

The nucleic acid can be translocated through the pore using a variety of mechanisms. For example, the nucleic acid can be electrophoretically translocated through the pore. Pore systems also incorporate structural elements to apply an electrical field across the pore-bearing membrane or film. For example, the system can include a pair of drive electrodes that drive current through the pores. Additionally, the system can include one or more measurement electrodes that measure the current through the pore. These can be, for example, a patch-clamp amplifier or a data acquisition device. For example, pore systems can include an Axopatch-1B patch-clamp amplifier (Axon Instruments, Union City, Calif.) to apply voltage across the bilayer and measure the ionic current flowing through the nanopore. The electrical field is sufficient to translocate a nucleic acid through the pore. As will be understood, the voltage range that can be used can depend on the type of pore system being used. For example, in some embodiments, the applied electrical field is between about 20 mV and about 20,000 mV.

In some embodiments, characteristics of the macromolecule can be determined based on the effect of the macromolecule on a measurable signal when interacting with the device. To illustrate, in some embodiments, the portion(s) of the macromolecule that determine(s) or influence(s) a measurable signal is/are the portions(s) residing in the constriction region (eg: the three-dimensional region in the interior of the pore with the narrowest dimension). Depending on the length of the constriction region, the portion(s) of the macromolecule that influence the current output signal, can vary. The output signal produced by the pore system is any measurable signal that provides a multitude of distinct and reproducible signals depending on the physical characteristics of the macromolecule. For example, the ionic current level through the pore is an output signal that can vary depending on the particular portion(s) of macromolecule residing in the constriction region of the device. As the macromolecule translocates in iterative steps (e.g., linearly, subunit by subunit through the pore), the current levels can vary to create a trace, or “current pattern,” of multiple output signals corresponding to the contiguous sequence of the nucleic acid subunits. This detection of current levels, or “blockade” events have been used to characterize a host of information about the structure of the nucleic acid passing through, or held in, a pore in various contexts.

In general, a “blockade” is evidenced by a change in ion current that is clearly distinguishable from noise fluctuations and is usually associated with the presence of an analyte molecule, e.g., one or more portions of the macromolecule, within the pore. The strength of the blockade, or change in current, will depend on a characteristic of the portions(s) of macromolecule present. Accordingly, in some embodiments, a “blockade” is defined against a reference current level. In some embodiments, the reference current level corresponds to the current level when the pore is unblocked (i.e., has no analyte structures present in, or interacting with, the pore). In some embodiments, the reference current level corresponds to the current level when the pore has a known analyte (e.g., a known nucleic acid subunit) residing in the pore. In some embodiments, the current level returns spontaneously to the reference level (if the pore reverts to an empty state, or becomes occupied again by the known analyte). In other embodiments, the current level proceeds to a level that reflects the next iterative translocation event of the macromolecule through the constriction, and the particular portion(s) of macromolecule residing in the pore change(s).

In some embodiments, the signal is generated by measuring an electrical property across a pair of electrodes that are situated within, or sufficiently near the constriction, such that a body translocating through said constriction also translocates between the electrode gap formed by said electrodes. The term “electrode,” as used herein, generally refers to a material or part that can be used to measure electrical signal. In some situations, electrodes can be disposed in the constriction and be used to measure the current across the constriction. The electrical signal can be a tunneling current. Such a current can be detected upon, e.g., the translocation of a macromolecule through the electrode gap, or a presence or absence of the macromolecule or a portion thereof within the electrode gap. In some cases, a sensing circuit coupled to electrodes provides an applied voltage across the electrodes to generate a current. As an alternative or in addition to, the electrodes can be used to measure and/or identify the electric conductance associated with the macromolecule, or portion there-of. In such a case, the tunneling current can be related to the electric conductance.

Electrode Gap. The term “electrode gap,” as used herein, generally refers to the region between electrodes that are situated within, or sufficiently near the constriction of a constriction device, such that a body translocating through said constriction also translocates through said electrode gap. The electrode gap may be disposed adjacent or in proximity to a sensing circuit or an electrode coupled to a sensing circuit. In some examples, an electrode gap has a characteristic width on the order of 0.1 nanometers (nm) to about 1000 nm.

The signals can be any types of electrical signals generated upon the passage of the macromolecule through the one or more electrode gaps, e.g., voltage, current, tunneling current, conductance, power, inductance, reactance, phase-shift etc. The electrical signals can comprise tunneling current when tunneling electrodes are utilized, and a measurement device can be employed for measuring tunneling current generated upon the passage of portion(s) of the macromolecule through the electrode gap(s). In some cases, a measurement device (or measurement unit) may be provided to measure the signal. The measurement device may comprise an ammeter, a current mirror, sense-measurement-unit (SMU), or any other current measurement or amplification approach, and an approach for quantifying the current, which may include an analog to digital converter (ADC), a delta sigma ADC, a flash ADC, a dual slope ADC, a successive approximation ADC, an integrating ADC, or any other appropriate type of ADC. The ADC may have a linear relationship between its output and the input, or may have an output which is tuned to the particular current levels which may be expected for a particular nucleic acid and the utilized electrode pair's physical and material manifestation. The response may be fixed, or may be adjustable, and may be adjustable particularly in conjunction with different outputs associated with the macromolecule's physical configuration.

The sense circuitry may generate its own current, voltage, power or combination there of. The generated current, voltage, and/or power may be constant, fluctuate with a constant frequency, fluctuate with a varying frequency, or fluctuate randomly, fluctuate based on a desired waveform, and/or fluctuate based on feedback mechanism.

The sense circuit may be on, or off the device, or a combination there-of.

Translocation. The terms “translocation” or “translocate,” as used herein, generally refers to the movement or containment of a macromolecule through a constriction region of a constriction device. The movement can occur in a defined, fixed, alternating, or a random direction. The movement or containment is at least partially controlled by a translocation force applied on said molecule. For clarity, in some embodiments, a translocation process results in only a portion of the molecule translocating a constriction device. For example: to translocate half the length of the molecule, and then reverse back. In addition, in some embodiments, a translocation process may include at least one time duration of no movement through the constriction region. For example, a translocation process wherein half the length of the molecule is translocated through a constriction device, and then stops for a period of time, and then continues movement. Used herein, the molecule is “translocating” a constriction device at any point in time in which the molecule is contained within said device, regardless of its final state, or if said molecule is in a state of movement relative to the constriction region.

Porous Material. A “porous material” is any composition of solid, or semi-solid matter that is porous in nature. In some embodiments, it may be a gel, formed by cross-linking a gelling agent. In some embodiments, it may be an artificial gel, manufactured with either random, or controlled pore sizes. The porous material may be fluidic device channel in which there are patterned physical obstacles that between them have openings, for example: a collection of pillars. The pillars may be of consistent, random, or distribution of sizes. The pillars may be arranged in a regular, planned, or random manner. The porous material may be a collection of packed beads or packed isolated objects, such that the space between the beads or objects provides for the porous nature. The beads or isolated objects may be of consistent, random, or distribution of sizes. The packing can be regular or random. In some embodiments, the porous material may be a material that is grown, etched, or deposited [Plawsky, 2009]. The material may be organic, inorganic, or a combination there-of. For the purpose of this document, the porous film should have at least a subset of pores (or openings) that are within the range from 50 microns to 50 nm in size.

Gels. “Gels” are defined as a substantially dilute or porous system composed of a “gelling agent” that has been cross-linked (“gelled”). Non-limiting examples of gels include agarose, polyacrylamide, hydrogels [Caló), 2015], DNA gels [Gačanin, 2020]. In the context of this document, a gel and a semi-gel are equivalent, where-by a semi-gel is a gel with incomplete cross-linking and/or low concentration of the gelling agent.

Methods of Physical Mapping the Feature Density Content of a Long Nucleic Acid Molecule with a Constriction Device

In the following set of embodiments, we describe methods of generating a linear physical map from a long nucleic acid molecule being interrogated by a constriction device, in which the linear physical map represents a genomic feature density profile, or dynamic conformational shift or change, along the major axis of the molecule. In some embodiments, the long nucleic acid molecule has bound to it at least one of at least one type of a labelling body. In some embodiments, the long nucleic acid molecule has no labeling bodies bound to it. In all cases, the detected signal as a function of time can be processed into a genomic or structure feature density or conformational change binned along the length of the major axis of the long nucleic acid molecule.

The feature of interest can be any genomic or structure (see definitions on “higher order nucleic acid structure”) content within the long nucleic acid molecule whose average normalized density per genomic length bin (in nanometers or microns) may vary along the major axis of said molecule. For example, the proportion of A-T base pairs within a 5 nm length of the long nucleic acid molecule. In another example, the proportion of nucleotides that are methylated within a 25 nm length of the long nucleic acid molecule. In another example, the proportion of 2-bp sequences that are 5′-AT-3′ within a 30 nm length of nucleic acid. In another example, the proportion of nucleic material contained in nucleosomes within a 50 nm length of the long nucleic acid molecule. In another example, the proportion of recognition sites within a 75 nm length of the long nucleic acid molecule. In another example, the proportion of nucleic material contained in TADs within a 100 nm length of the long nucleic acid molecule. In another example, the proportion of nucleic material contained in nucleic loops within a 100 nm length of the long nucleic acid molecule. In another example, the proportion of nucleic acid material contained in cohesin-dependent chromatin loops within a 100 nm length of the long nucleic acid molecule. In another example, the proportion of nucleic acid material bound to a cohesin complex within a 100 nm length of the long nucleic acid molecule. In another example, the interphase chromatin organization is rapidly lost in a condensin-dependent manner when progressing towards prophase, and arrays of consecutive 60-kilobase (kb) loops are formed. During prometaphase, ˜80-kb inner loops are nested within-400-kb outer loops. The loop array acquires a helical arrangement with consecutive loops emanating from a central “spiral staircase” condensin scaffold. In another example, The size of helical turns progressively increases to −12 megabases during prometaphase. For embodiments where-by the long nucleic acid molecule is largely without higher order structure such that path along the length of the nucleic acid polymer and the major axis are one of the same, the length in nanometers can be converted to length in basepairs using a conversion appropriate for the conditions in which the molecule is interrogated. In some embodiments, the translocation speed of the molecule through the constriction region can be estimated by signal processing to elucidate a component of the signal from single nucleotides.

In all embodiments, the unit of genomic length bin can vary depending on the size of constriction device used, the relative frequency and rarity of the feature of interest, the choice of labeling body type, and methods of their use, including translocation speed. In some embodiments, the bin is about 1 nm, or about 2 nm, or about 5 nm, or about 7 nm, or about 10 nm, or about 12 nm, or about 15 nm, or about 20 nm, or about 25 nm, or about 30 nm, or about 35 nm, or about 40 nm, or about 50 nm, or about 60 nm, or about 75 nm, or about 100 nm, or about 125 nm, or about 150 nm, or about 200 nm, or about 250 nm, or about 500 nm, or about 750 nm, or about 1000 nm, or about 1250 nm, or about 1500 nm, or about 2000 nm, or about 2500 nm.

FIG. 4(A) demonstrates an embodiment method for generating a linear physical map where-by the feature of interest is associated with one type of labeling body (407), such that there is a correlation along the length of the major axis of the long nucleic acid molecule (406) between the density of the labelling bodies, and the density of the features.

For brevity, in this drawn embodiment (FIG. 4(A)), the constriction device is a current blockade constriction device of the type previously described for FIG. 3(A) in the definition of “constriction device”. However, the other previously described constriction devices described can also be used as variations of this embodiment method. Here in this drawn embodiment, both the translocation (404) of the long nucleic acid, and the measured current through the constriction region (403) are performed by the SMU (402).

FIG. 4(B) demonstrates a measured current trace (414) from the device shown in FIG. 4(A) as the long nucleic acid molecule 406 translocates the constriction region. The trace plots the measured signal (411) vs the time of the measurement (417). In this embodiment, the long nucleic molecule is translocated through the constriction region at approximately a consistent velocity. However, in some embodiments, the translocation speed may be adjusted, stopped, or reversed. As the molecule enters the constriction region, the current decreases (412) due to the current blockade effect. As an example, a local region along the length of the long nucleic acid molecule with a high labeling body density (405) is demonstrated as a localized reduction in measured current (413). When the molecule exits the constriction region (415), the measured current returns to its original baseline (416) of an un-obstructed constriction region.

FIG. 4(C) represents a processed transformation of the signal shown in FIG. 4(B) in which genomic length bins (423) of a normalized density (421) are plotted in nanometers (426), in which the length of the long nucleic acid molecule's major axis is shown (425). Here, each bin can contain up to a maximum of 100% occupancy (424) of a normalized feature density within the bin. Again, as an example, a local region along the length of the long nucleic acid molecule with a high labeling body density (405) is demonstrated as localized collection of bins with high density (422).

In some embodiments, the relationship between the genomic feature density and labelling body is a positive correlation along the length of the long nucleic acid molecule's major axis, for example a labelling body type that is more likely to bind to a region of the macromolecule with a high density of said features. In some embodiments, the relationship between the genomic feature density and labelling body is a negative correlation along the length of the long nucleic acid molecule's major axis, for example a labelling body type that is more likely to bind to a region of the macromolecule with a low density of said features.

In some embodiments, the value given to each bin is exclusively derived from processing signal data from at least one time period of measurements by the constriction device, such that no interrogation signal data point is used for more than one bin. In some embodiments, multiple bins may use the same signal data points, for example if a weighted time-averaging is performed, or if signal processed is used, such as to accommodate for nearest-neighbor factors along the length of the long nucleic acid molecule.

In all embodiments where-by a type of label body is bound to the long nucleic acid molecule, the label body will alter the measured signal of the molecule as it is interrogated by the constriction device, compared to the signal of the same molecule with no such a label body when interrogated by the same constriction device. In some embodiments, different labelling body types may generate similar signals in a constriction device. In some embodiments, different labelling body types may generate different signals in a constriction device. In some embodiments, for a fixed translocation force, a labelling body may reduce the translocation speed of the long nucleic molecule when said body, bound to the molecule, is being interrogated by the constriction device. In some embodiments, for a fixed translocation force, a labelling body may increase the translocation speed of the long nucleic molecule when said body, bound to the molecule, is being interrogated by the constriction device.

For all embodiments, the translocation force can include any of the following, or combinations there-of: electrokinetic, electrophoretic, electroosmotic, capillary, pressure.

In some embodiments, multiple labelling body types are bound to the long nucleic acid molecule, in which at least two different body types may have a different signal.

In some embodiments, multiple labelling body types are bound to the long nucleic acid molecule, in which at least two different body types may have a similar signal.

In some embodiments, the relationship between the genomic feature and the labelling body weakly correlated, or weakly anti-correlated. For example, a method of generating a label body profile by first non-specifically labelling the nucleic acid and then selectively releasing label bodies in AT rich regions via partial melting to produce a correlation between labeling bodies and CG rich regions. However, if a small CG rich region is sandwiched by two large AT rich regions, the physical coupling may result in a loss of some or all labels within the small CG rich region.

In some embodiments, the translocation speed is modulated, including increased, decreased, reversed, stopped. In some embodiments, the modulation of the speed is based on a feed-back mechanism based on data from at least one constriction device. In some embodiments, the long nucleic acid molecule is fluorescently interrogated while also being interrogated by the constriction device. In this embodiment, at least one input to the feedback mechanism that controls the molecule translocation can include the fluorescent interrogation data. In some embodiments, at least a sub-set of fluorescent labelling bodies along the long nucleic acid molecule comprises a physical map.

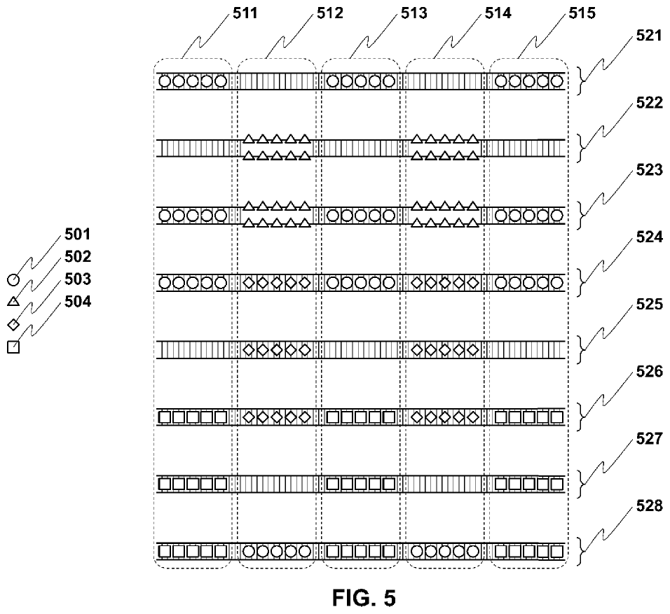

FIG. 5 demonstrates several non-limiting embodiments in which the feature density linear physical map comprises an AT/CG density linear physical map on long nucleic acid molecules (521 through to 528). Here, 501 represents a ds-DNA non-specific labeling body type (non-specific with respect to AT/CG content), 502 represents a ss-DNA labeling body type, 503 represents a ds-DNA AT-specific, or AT-rich specific labeling body type, and 504 represents a ds-DNA CG-specific, or CG-rich specific labeling body type. For all embodiments, 511, 513, and 515 represent regions along the long nucleic acid molecules where the CG content is relatively high (“CG rich” regions wherein the CG content is at least 51% of the genomic content), while 512 and 514 represent regions along the long nucleic acid molecules where the AT content is relatively high (“AT rich” regions wherein the AT content is at least 51% of the genomic content). For all embodiments where-by two different labelling body types are used to differentiate between CG-rich and AT-rich regions respectively, the two different labelling body types when bound to a long nucleic acid molecule generate distinct (from one another) signals when said molecule is interrogated by a constriction device.

The long nucleic acid molecules 521, 522, and 523 each comprises an AT/CG density linear physical map generated by a variation of the melt-map process (see “physical map” in definitions) wherein here, the labelling body type(s) used need not be fluorescent, as the embodiment methods use a constriction device for interrogation. For the molecule 521, the molecule is first non-specifically bound with a labelling body type 501, then melted to release the labelling body type 501 from the AT rich areas to produce an AT/CG density linear physical map. For the molecule 522, the molecule is partially melted, and while partially melted the labelling body type 502 is bound to the AT-rich single nucleic acid strands to produce an AT/CG linear physical map. For the molecule 523, the molecule is first non-specifically bound with a labelling body type 501, then melted to release the labelling body type 501 from the AT rich areas, which are then bound to by single-strand labelling body type 502, to produce an AT/CG linear physical map. Alternatively in another embodiment method, for the molecule 523, the molecule is partially melted, and while partially melted the labelling body type 502 is bound to the AT-rich single nucleic acid strands, the molecule is re-annealed, and then a double strand non-specific labelling body type 501, or a CG-specific labelling body type 504 is bound to the CG-rich regions, as double-stranding binding in the AT rich regions is degraded due to the presence of the single-strand labelling body types locally inhibiting re-annealing.

The long nucleic acid molecules 524, 525, 526, 527, and 528 each comprises an AT/CG density linear physical map generated by a variation in the competitive binding process (see “physical map” in definitions), however here the labeling bodies need not be fluorescent, as the molecules will be interrogated with a constriction device. For the molecule 524, the molecule is bound to by a non-specific labeling body type 501, and an AT-rich specific labeling body type 503, wherein within the AT-rich regions of the molecule, the second labelling body type will out-compete the first labelling body type for bonding, under the bonding conditions (temperature, reagent concentration, pH, buffer composition, etc), producing an AT/CG linear physical map. For the molecule 525, the molecule is bound to by an AT-rich specific labeling body type 503, producing an AT/CG linear physical map. For the molecule 526, the molecule is bound to by a CG-rich-specific labeling body type 504, and an AT-rich specific labeling body type 503, wherein the within the AT-rich regions of the molecule, the second labelling body type will out-compete the first labelling body type for bonding, and within the CG-rich regions of the molecule, the first labelling body type will out-compete the second labelling body for bonding, under the bonding conditions (temperature, reagent concentration, pH, buffer composition, etc), producing an AT/CG linear physical map. For the molecule 527, the molecule is bound to by an CG-rich specific labeling body type 504, producing an AT/CG linear physical map. For the molecule 528, the molecule is bound to by a non-specific labeling body type 501, and a CG-rich specific labeling body type 504, wherein the within the CG-rich regions of the molecule, the second labelling body type will out-compete the first labelling body type for bonding, under the bonding conditions (temperature, reagent concentration, pH, buffer composition, etc), producing an AT/CG linear physical map.

For all embodiments where-by two different labeling body types are used, and where-by each labeling body type identifies a CG-rich or AT-rich region respectively, in some embodiments, the physical map represents the ratio or relative proportion of the two body types along the length of the molecule's major axis. In some embodiments, the signal from each individual label body type is first processed, and then the ratio or the relative proportion of the two body types along the length of the molecule's major axis is determined. In some embodiments, this processing can include normalization, correcting for variation for translocation speed, correcting for variation in stretch, correcting for nearest-neighbor influence along the molecule, correcting for signal strength difference between the two label body types.

For all embodiments where-by two different labeling body types are used, and where-by each labeling body type identifies a CG-rich or AT-rich region respectively, the relative proportion of specific labelling body type within its respective associated region need not be 100% as drawn in FIG. 5. For example, a labeling body type 1 that identifies an AT-rich region and a labeling body type 2 that identifies a CG-rich region: within a AT-rich region, the proportion of type 1 labelling bodies and type 2 labelling bodies measured may be 100% and 0% respectively, or in some cases 90% and 10% respectively, or in some cases 80% and 20% respectively, or in some cases 70% and 30% respectively, or in some cases 60% and 40% respectively. In some cases, the label body type that associates with a particular region may in fact be in the minority of the measured label body types within that region, as is the case when one label body type has a high degree of non-specific binding. Thus, for example, a labeling body type 1 that identifies an AT-rich region and a labeling body type 2 that identifies a CG-rich region: within a AT-rich region, the proportion of type 1 labelling bodies and type 2 labelling bodies measured may be 40% and 60% respectively, or in some cases 30% and 70% respectively, or in some cases 20% and 80% respectively, or in some cases 10% and 90% respectively. In all cases, for a given labeling condition, a look-up table or function of measured relative proportion of type 1 and type 2 labels for a particular region can be used to determine the degree of “AT-rich”-ness and “CG-rich”-ness within said region.

Examples of non-specific double-strand labelling bodies (501) include: Intercalating molecules (including: Florescent Intercalating molecules, dimeric cyanine nucleic acid stain, POPO-1, BOBO-1, YOYO-1, JOJO-1, POPO-3, LOLO-1, BOBO-3, YOYO-3, TOTO-3 5F-203, 4′-Aminomethyltrioxsalen hydrochloride, 2-Amino-9H-pyrido[2-3-b]indole, Angelicin, (S)-tert-Butyl 1-(chloromethyl)-5-hydroxy-1H-benzo[e]indole-3(2H)-carboxylate, Carboplatin, Carmustine, CB 1954, Chlorambucil, Cryptolepine hydrate, Cyclophosphamide monohydrate, Fotemustine, Melphalan, Mitoxantrone dihydrochloride, Oxaliplatin, Procarbazine hydrochloride, Psoralen, Tirapazamine, Treosulfan, Trioxsalen), High-Mobility Group or HMG, Histones, Minor-groove binding proteins, RecA, Major-groove binding proteins, any fluorescently tagged variant there-of, any modified variant there-of

Examples of single-strand labelling bodies (502) include: Single-stranded binding proteins (SSBs), Replication protein A (RPA), RPA1, RPA2, RPA3, DNA replication associated factors and complex, DNA repairing associated factors and Complex, DNA transcription associated factors and complex, any fluorescently tagged variant there-of, any modified variant there-of.

Examples of AT-rich specific labelling bodies (503) include: netropsin, distamycin, Acridine homodimer bis-(6-chloro-2-methoxy-9-acridinyl)spermine, ACMA (9-amino-6-chloro-2-methoxyacridine), AT-selective DAPI (4′,6-diamidino-2-phenylindole), hydroxystilbamidine, Hoechst 33258, Hoechst 33342, Hoechst 34580, DB75, Pentamidine, Beneril, BAPPA, phytoestrogen tanshinone IIA, any fluorescently tagged variant there-of, any modified variant there-of.

Examples of CG-rich specific labelling bodies (504) include: 7-AAD (7-aminoactinomycin D), Actinomycin D. Echinomycin, Mithramycins (MTMs), Lurbinectedin, any fluorescently tagged variant there-of, any modified variant there-of.

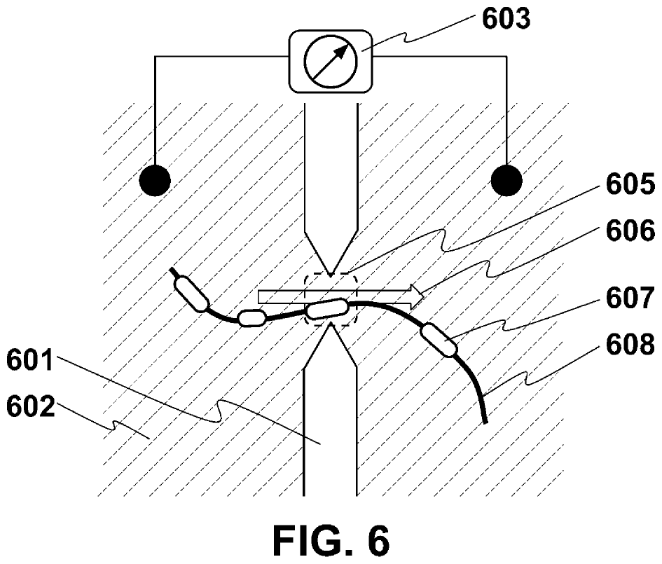

FIG. 6 represents another embodiment, wherein a AT/CG density linear physical map is a “bubble map”, generated without a labelling body. Here the long nucleic acid molecule (604) is interrogated by a constriction device (601), in which at least a portion of the molecule is in a partially melted state, forming de-natured single-strand bubbles (607) in regions of high AT density. In this embodiment, the signal generated from a de-natured region of the molecule when in the constriction region (605) will generate a different signal had that region of the molecule been fully hybridized, thus allowing for differentiation between de-natured (AT rich) and hybridized (CG-rich) regions along the length of the long nucleic acid molecule's major axis.

Non-limiting examples of denaturing conditions include any of the following, including combinations there-of: temperature, ionic concentration, buffer conditions, pH.

In some embodiments, the denaturing conditions can be changed on-the-fly such that nucleic acid's partially de-natured profile can be modified by adjusting the degree of denaturation. In some embodiments, this modulation can be controlled by a feedback system at least in part informed by the constriction device signal, so as to allow for tuning of the denaturation profile based on the genome, or optimization of denaturing signal for a particular genomic feature of interest. In some embodiments, at least a portion of the long nucleic molecule may be interrogated at least twice, each with different de-naturing conditions. For example, a small CG-rich island sandwiched between two larger AT-rich regions may is de-natured at one temperature, but is hybridized while maintaining the denatured state of the AT-rich regions at a lower temperature. Alternatively, a small AG-rich region sandwiched between two CG-rich regions may remain hybridized at one temperature, but denature while still maintaining the hybridized state of the CG-rich regions at a higher temperature. Thus interrogating over a range of denaturing conditions allows for elucidating finer resolution of the AT and CG rich regions.

In some embodiments, a long nucleic acid molecule, in a partially melted state, has at least a portion of the molecule's length along the major axis interrogated by a constriction device at least one time, at a temperature of about 24° C., or about 26° C., or about 28° C., or about 30° C., or about 32° C., or about 34° C., or about 36° C., or about 38° C., or about 40° C., or about 42° C., or about 44° C., or about 46° C., or about 48° C., or about 50° C., or about 52° C., or about 54° C., or about 56° C., or about 60° C., or about 62° C., or about 64° C., or about 66° C., or about 68° C., or about 70° C., or about 72° C., or about 74° C., or about 76° C., or about 78° C., or about 80° C., or about 82° C., or about 84° C., or about 86° C., or about 88° C., or about 90° C., or about 92° C., or about 94° C., or about 96° C., or about 98° C.

For brevity, in this drawn embodiment (FIG. 6), the constriction device is a current blockade constriction device of the type previously described for FIG. 3(A) in the definition of “constriction device”. However, the other previously described constriction devices described can also be used as variations of this embodiment method. Here, both the translocation (606) of the long nucleic acid, and the measured current through the constriction region (605) are performed by the SMU (603).

For all embodiments, the signal from the constriction device as the long nucleic acid molecule is interrogated can be monitored, and the conditions under which the interrogation occurs can be adjusted. Such conditions include translocation speed (including rate, stopping, and reversing), temperature, pH (each side of the constriction independently), ionic concentration (each side of the constriction independently), buffer composition (each side of the constriction independently), reagent concentration (each side of the constriction independently), and reagent composition (each side of the constriction independently).

For all embodiments, the signal from the constriction device as the long nucleic acid molecule is interrogated will be processed to generate a consensus feature density profile along the length of the major axis of the long nucleic acid molecule which represents a linear physical map. Processing to generate this profile may include filtering of noise, removal of signal generated by the nucleic acid itself, adjustments or corrections for variation in the translocation speed or force, signal processing, pattern recognition, comparison to a reference (including to correct and filter), nearest-neighbor effects along the molecule, machine-learning techniques, frequency domain analysis, sampling, heuristic tree algorithm, Bayesian network, hidden Markov model, or conditional random field. In particular, multiple reads of the same portion of the long nucleic acid molecule can be performed to aid in filtering of noise.

In some embodiments, a multitude of signals from the constriction device, or at least a portion of the feature density profile, or at least a portion of the consensus feature density profile can be analyzed in the frequency domain. In some embodiments, frequency is defined as the number per unit of time, for example, the number of signals measured per unit of time. In some embodiments, frequency is defined as the number per unit of absolute or genomic distance (eg: nm or bp), for example, the number of bins per 10 microns, or the number of bins per 100,000 bp. In some embodiments, the frequency domain analysis is used to generate a unique frequency barcode. In some embodiments, the frequency barcode is compared to a reference.

In all embodiments, the long nucleic acid molecule can also be fluorescently interrogated. In some embodiments, the fluorescent interrogation occurs during a time point in which said molecule is also being interrogated by the constriction device. In some embodiments, the long nucleic acid molecule is bound with fluorescent labeling bodies that provide for a linear physical map. In the preferred embodiment, the fluorescent labeling bodies also provide for a linear physical map that can be interrogated by the constriction device. In the preferred embodiment, the spatial fluorescent linear physical map is interrogated a multitude of times by the fluorescent interrogation device, and the time point of each data set can be coordinated with the time point of the constriction device. In some embodiments, such coordination allows for a registration of where along the major axis of the long nucleic molecule (with respect to the fluorescent linear physical map) a measured signal with the constriction device is taken. In some embodiments, the fluorescent data allows for a determination of the long nucleic acid molecule's velocity at a particular time point of the constriction device interrogation. The velocity may be the global (average) speed of the molecule's mass, or the particular translocation speed of the portion of the molecule in the constriction device, or both. In some embodiments, the fluorescent data allows for a determination of the long nucleic acid molecule's stretch at a particular time point of the constriction device interrogation. The stretch may be the global (average) stretch (extension) of the molecule, or the particular stretch of the portion of the molecule in the constriction device, or both. All such data can be used to provide contextual location information to the constriction data, or to signal process the constriction device data, or both.

For all embodiments, once a feature density linear physical map has been generated for the long nucleic acid molecule, this map can then be compared to a reference in order to identify the molecule or features of interest within the molecule. These features may include unique patterns that can be used to identify and/or analyze the originating genome, the originating chromosome, a gene, a break-point, a regulatory region, a disease-associated region, a structural variation, a copy number, a deletion, a phenotype, a phase, a telomere, a sub-telomere, a centromere, a sub-centromere.

For all embodiments, after an interrogation by a constriction device to generate a feature density physical map is complete, in some embodiments the molecule is then further processed. In some embodiments, this processing comprises sequencing, amplification, a reaction with an enzyme. In some embodiments, the processing is done on, or off the fluidic device that comprises the constriction device. In some embodiments where-by the molecule is extracted from the fluidic device, it is first encapsulated in a droplet. In some embodiments, the droplet is a water-in-oil droplet, or a water-in-oil-in-water droplet. In some embodiments, a decision to further process the molecule is based at least partially on an analysis of the molecule's physical map.

Devices and Methods for Interrogating Higher Order Nucleic Acid Structure with a Constriction Device

The following set of embodiment devices and methods pertains to analysis of a long nucleic acid molecule that comprises at least one higher order nucleic acid structure (or “structure”) by interrogation with at least one constriction device. Here, the structure(s) itself provides the signal which is measurably different from signal generated by interrogating with a constriction device a similar long nucleic acid molecule with no such structure(s).

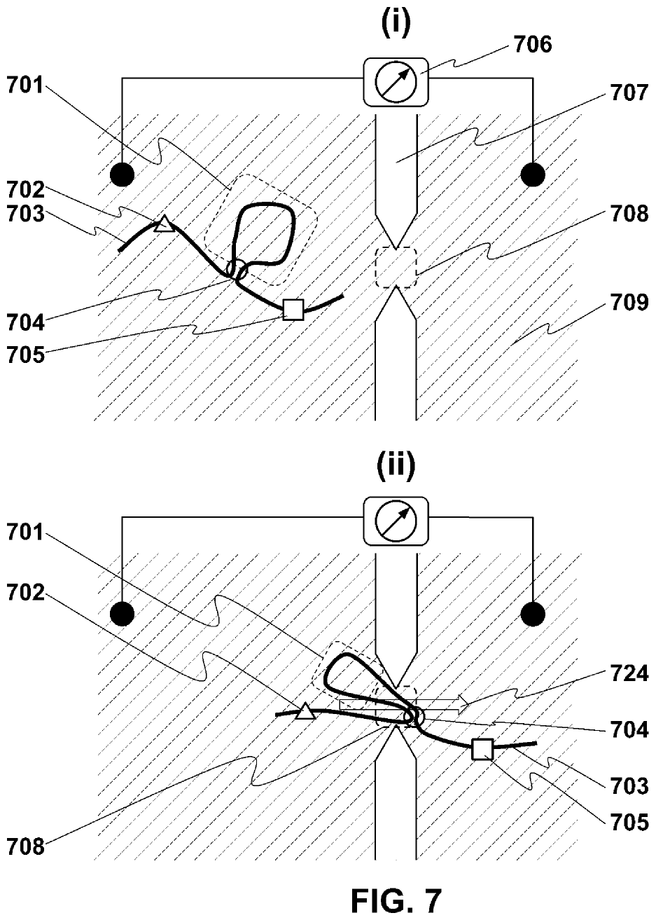

In one embodiment, shown in FIG. 7, a long nucleic acid molecule (703) with a transcription complex (702) is interrogated with a constriction device (707). Here, the physical configuration of the nucleic acid along with the proteins that make up the complex provide a signal as the nucleic acid is interrogated by the constriction device. In this drawn embodiment, the complex consists of a cohesin complex, resulting in a nucleic acid loop (701). Such a signal can be processed to provide information with respect to the size of the loop, and the locations of the proteins with respect to each other. In FIG. 7(i) the molecule is brought towards the constriction region (708) under control of the SMU (706) in electrical contact with the solution (709) that fluidically connects both sides of the constriction. Later in time (ii), the molecule enters the constriction region, and the molecule with its structure interact with the constriction region. The interaction may be one of a reduction in the molecule's mobility as the structure translocates (724) through the constriction, or a modulation in the measured constriction device signal as a function of what portion(s) of the molecule or what portion(s) of the structure(s) are present in the constriction region while the signal measurement(s) are made. In some embodiments, the physical conformation, or physical composition, or physical dimensions of the structure is further interrogated by alternating the direction of translocation or ceasing the translocation, allowing the structure to twist, alternate, turn, re-position, or relax while inside the constriction region. In particular, in some embodiments where-by the structure includes at least one loop which can re-orientated via an applied force relative the major axis of the long nucleic acid molecule, the structure is interrogated by translocating the molecule in one direction, resulting in one orientation of the loop in the constriction region, and then the translocation direction is reversed, allowing for a different orientation of the loop in the constriction region. In some embodiments, the structure is interrogated by the constriction device by completely translocating the structure through the constriction region, and then interrogating the structure at least a second time by reversing the direction of the translocation.

In some embodiments, at least one sequence specific labelling bodies (705, 702) are bound to the nucleic acid to provide landmarks which can be used to identify where in the genome such a structure is located. In other embodiments, the long nucleic acid molecule is bound with labelling bodies to generate a linear physical map to allow for identification of the long nucleic acid molecule by comparison to a reference. In some embodiments the linear physical map is an AT/CG density linear physical map. In some embodiments, the long nucleic acid molecule is interrogated under conditions that partially melt at least a portion of the molecule to provide an AT/CG density linear physical map.

For brevity, in this drawn embodiment (FIG. 7), the constriction device is a current blockade constriction device of the type previously described for FIG. 3(A) in the definition of “constriction device”. However, the other previously described constriction devices described can also be used as variations of this embodiment method. Here, both the translocation (724) of the long nucleic acid, and the measured current through the constriction region (708) are performed by the SMU (706).

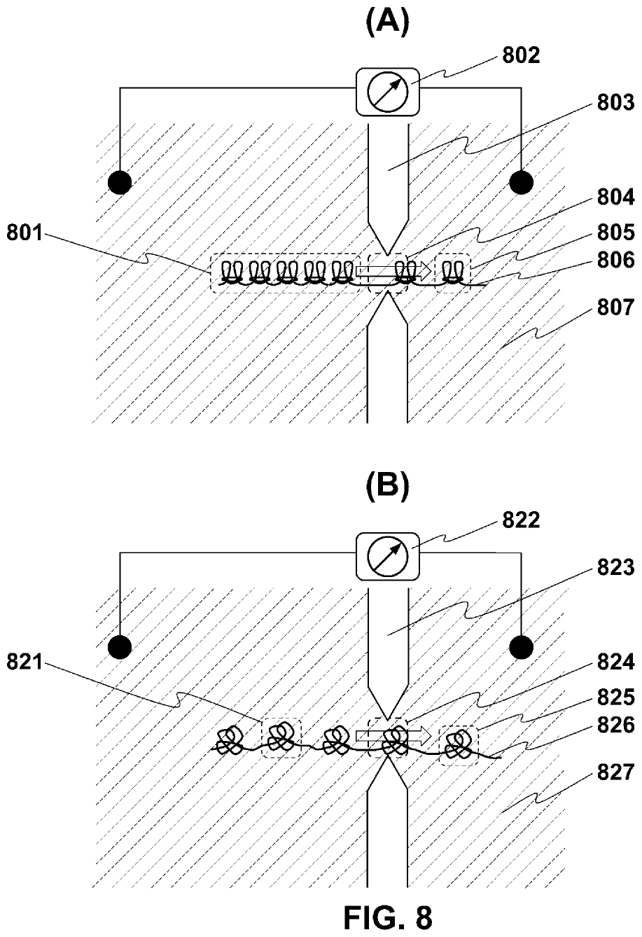

In another embodiment, shown in FIG. 8(A), a long nucleic acid molecule (806) with nucleosomes (805) is interrogated in the constriction region (804) of a constriction device (803) such that the number, spacing, density or nature of the nucleosomes can be determined. In particular, the regional boundary between the heterochromatin (801) and the euchromatin can be determined.

For brevity, in this drawn embodiment (FIG. 8(A)), the constriction device is a current blockade constriction device of the type previously described for FIG. 3(A) in the definition of “constriction device”. However, the other previously described constriction devices described can also be used as variations of this embodiment method. Here, both the translocation of the long nucleic acid, and the measured current through the constriction region (804) are performed by the SMU (802).

In another embodiment, shown in FIG. 8(B), a long nucleic acid molecule (826) with topologically associating domains (TADs) (825) is interrogated in the constriction region (824) of a constriction device (823) such that the number, spacing, density, size, orientation (with respect to the molecule's major axis), loop count per TAD, or nature of the TADs can be determined.

For brevity, in this drawn embodiment (FIG. 8(B)), the constriction device is a current blockade constriction device of the type previously described for FIG. 3(A) in the definition of “constriction device”. However, the other previously described constriction devices described can also be used as variations of this embodiment method. Here, both the translocation of the long nucleic acid, and the measured current through the constriction region (824) are performed by the SMU (822).

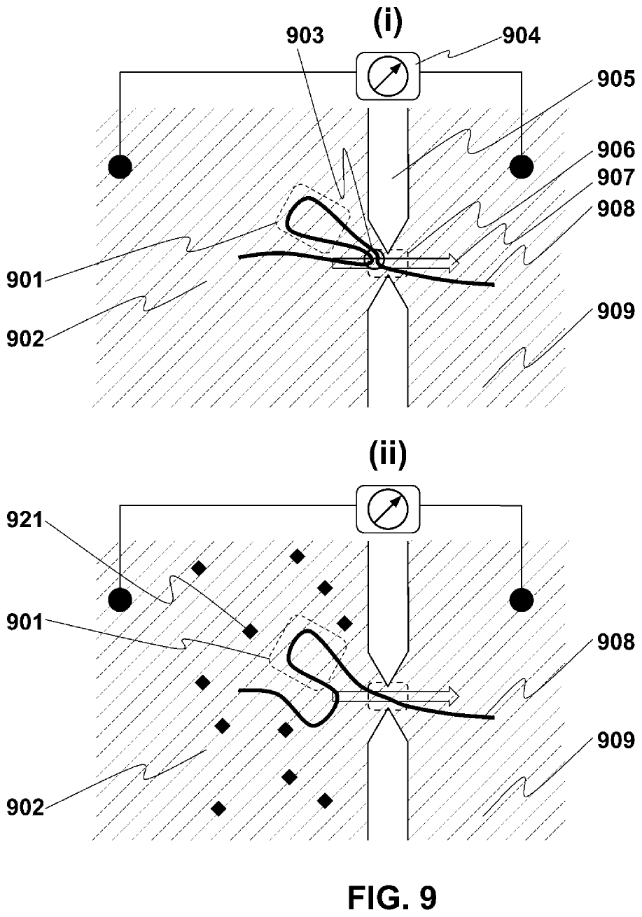

In another embodiment, shown in FIG. 9, (i) a long nucleic acid molecule (908) is partially translocated (907) through a constriction region (906) of a constriction device (905), however for a particular translocation force applied on the molecule, said molecule is unable to completely translocate through the constriction region due to the physical conformation of the structure (903) on said molecule. In this particular embodiment drawing, the structure is a cohesin complex resulting in a nucleic acid loop (901). At a later point in time, (ii) on at least one side of the constriction region, a digestive enzyme (901) is introduced that can digest, or partially digest, the structure and free the loop (901). In this embodiment drawing, the enzyme is only introduced on the originating side (902) of the constriction region. With the structure now modified, for the same particular translocation force applied on the molecule, said molecule is now able to translocate through the constriction region of the constriction device.

In other embodiments, the enzyme is introduced on the exit side (908) of the constriction region, or both sides.

In some embodiments, the enzyme does not digest the nucleic acid or structure, but nicks the long nucleic acid molecule or structure. In some embodiments, the digestion, or partial digestion of the structure results in a physical re-configuration of said structure. For example, a multi-loop structure may have the loop count reduced by at least one loop. In another example, at least two loops may join to form a single loop.

In another embodiment, an enzyme reagent is already present on the exit side of the constriction device, such that upon translocating through the constriction device, at least a portion of the long nucleic acid molecule or a portion of a structure that molecule comprises is digested, partially-digested, or nicked. After digestion or nicking, the molecule is then re-interrogated in the same constriction device, or a different constriction device.

In some embodiments, the enzyme is a specific enzyme, selected to digest or nick a specific target protein. In some embodiments, the enzyme is selected to digest or nick a specific sequence of nucleic acid sequence.

In some embodiments, the environmental or solution conditions are modulated to disrupt the structure. These conditions can include pH, temperature, a reagent concentration, or ionic strength or conductivity of the buffer. In some embodiments where-by the solution conditions include a reagent concentration or composition, the reagent comprises a labeling body, a DNA binding protein, a polymerase, a nucleotide, a modified nucleotide or a photo-activated reagent.

In the preferred embodiment, a change in the mobility of a long nucleic acid molecule with at least one structure through a constriction region, to a fixed translocation force, before and after exposure to an enzyme, or environment condition, or solution condition, provides information as to the nature of the structure. In some embodiments, the mobility increases after exposure. In some embodiments, the mobility decreases after exposure.

In some embodiments, at least one enzyme is bound to the constriction device. In some embodiments, the enzyme is bound to the constriction region.

For brevity, in this drawn embodiment (FIG. 9), the constriction device is a current blockade constriction device of the type previously described for FIG. 3(A) in the definition of “constriction device”. However, the other previously described constriction devices described can also be used as variations of this embodiment method. Here, both the translocation of the long nucleic acid, and the measured current through the constriction region (906) are performed by the SMU (904).

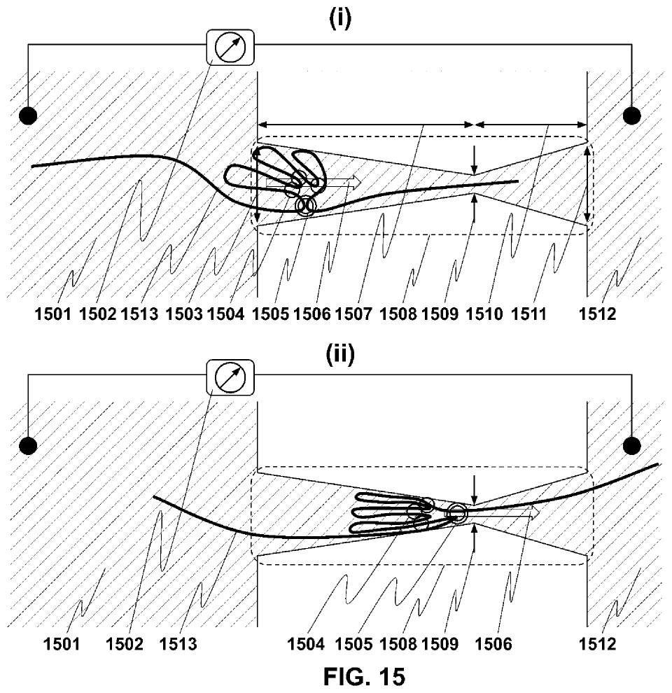

FIG. 15 demonstrates a constriction device wherein the constriction region (1508) is elongated along the translocation axis such that there is a gradual transition from the inlet of the constriction region with an inlet dimension (1503) to the constriction region critical dimension (1509), wherein the length of this transition (1507) is long enough to physically enclose the structure of interest. In addition, in this particular drawn embodiment, there is also gradual transition from the constriction region critical dimension (1509) to the outlet of the constriction region with an outlet dimension (1511), wherein the length of this transition (1510) is long enough to physically enclose the structure of interest. In some embodiments, there is only an inlet transition. In some embodiments, there is only an outlet transition.

In the drawn embodiment of FIG. 15 (i), a translocation force (1506) generated by an SMU (1502), in electrical connection with the inlet fluidic chamber (1501) and the outlet fluidic chamber (1512), is applied to a long nucleic acid molecule (1513) with at least one structure, such that the molecule is brought into the constriction region (1508) wherein the physical conformation of the molecule and its structure are deformed via interaction with said constriction region. Furthermore, the deeper into the constriction, as shown in FIG. 15 (ii) at a later time point, the greater the confinement on the molecule and structure, and with it, a further change in said structure's physical conformation.

In this particular drawn embodiment, the structure consists of three condensin I (1504) nucleic acid loops, all bound together by a single condensin II (1505).

In some embodiments, the interrogation of the structure in the constriction device comprises fluorescent monitoring via at least one labelling body on the long nucleic acid molecule or structure of the molecule's physical position within the transition region as a function of different translocation forces. In some embodiments, the interrogation of the structure in the constriction device comprises modulating the translocation force such that at least a portion of the structure is contained in the inlet transition, and at least a portion of the structure is contained in the outlet transition.

In some embodiments, the inlet or outlet transition length (1507 and 1510 respectively) is 100 nm or longer, or 250 nm or longer, or 500 nm or longer, or 1000 nm or longer, or 2000 nm or longer, or 5000 nm or longer. In some embodiments, the inlet or outlet entrance defining dimension (1503 and 1511 respectively) has a length that is at least 1.5 times or greater the constriction region critical dimension (1509), or 2 times or greater, or 3 times or greater, or 5 times or greater, or 10 times or greater, or 50 times or greater, or 100 times or greater.

The gradual reduction in confinement region dimensions from the inlet (1503) to critical dimension (1509) imposes an entropic force that acts on nucleic acids confined in this region and pulls them away from the narrowest portion of the constriction region, where the critical dimension is located. In some embodiments, the local density of nucleic acid occupying the constriction region can be measured by uniform fluorescent labeling of the nucleic acid combined with fluorescence imaging of the constriction region. This measured fluorescent density decreases as the molecule translocates deeper in the narrower region. In the particular embodiments wherein the critical dimension is 100 nm or less, it is improbable for more than one strand of nucleic acid to be present at once without a sufficiently large applied translocation force. A constriction device can be calibrated to measure the typical intensity vs. distance profile observed for a combination of device dimensions, buffer conditions, external electric field and other sources of hydrodynamic drag such as pressure driven flow. The overall intensity of the profile can vary with fluorophore: nucleotide ratio, temperature and excitation and detection efficiencies, but the relative shape of the profile is invariant to these perturbations.

When topologically looped nucleic acids are pulled deeper into the narrower portion of the constriction region, the local concentration of nucleic acid increases. This is detectable in several complimentary ways. Fluorescence imaging shows a local increase in nucleic acid density inside the reducing constriction region, and this can be detected by a change in the shape of the intensity vs. position profile, or by an absolute increase in fluorescence intensity. At the wider portions of the constriction region as it is more difficult to distinguish locally interacting portions of loops from distal regions of nucleic acid that happen to be gyrating in close proximity, especially after electrophoretic force of hydrodynamic force resulting from electroosmotic flow has acted to concentrate nucleic acids within the constriction region. As the constriction region narrows, it is easier to detect above average levels of nucleic acids that result from looped structures moving together. In this regime a simple loop structure results in 3× fluorescent intensity of a single strand and this continues up until the origin of the loop, where intensity suddenly drops to that expected of a single strand. More complicated loops, for example those relating to nested loop arrays organized by Condensin II and Condensin I, do not show such simple patterns, but nonetheless when observing from the widest part of the constriction to the narrowest, there is a local increase of fluorescence followed by a sudden drop as the loop origin is reached.

The extent of the looping structure can be further estimated by applying an external force (eg: electrophoretic or hydrodynamic drag from electroosmotic flow) and letting the nucleic acid come to rest inside the tapered constriction region. The origin of the loop is located as mentioned above and the position is measured in relation to the geometry of the constriction region. Under identical external forces, larger loops will proceed further toward the constriction critical dimension than smaller loops. The translocation force generated by the SMU (1502) is then ramped up until the loop structure completely translocates the constriction region, and a trace of voltage and current pertaining to the event is recorded, both of which reflect the size and composition of the looped structure.

For brevity, in this drawn embodiment (FIG. 15), the constriction device is a current blockade constriction device of the type previously described for FIG. 3(A) in the definition of “constriction device”. However, the other previously described constriction devices described can also be used as variations of this embodiment method. Here, both the translocation (1506) of the long nucleic acid, and the measured current through the constriction region (1508) are performed by the SMU (1502).

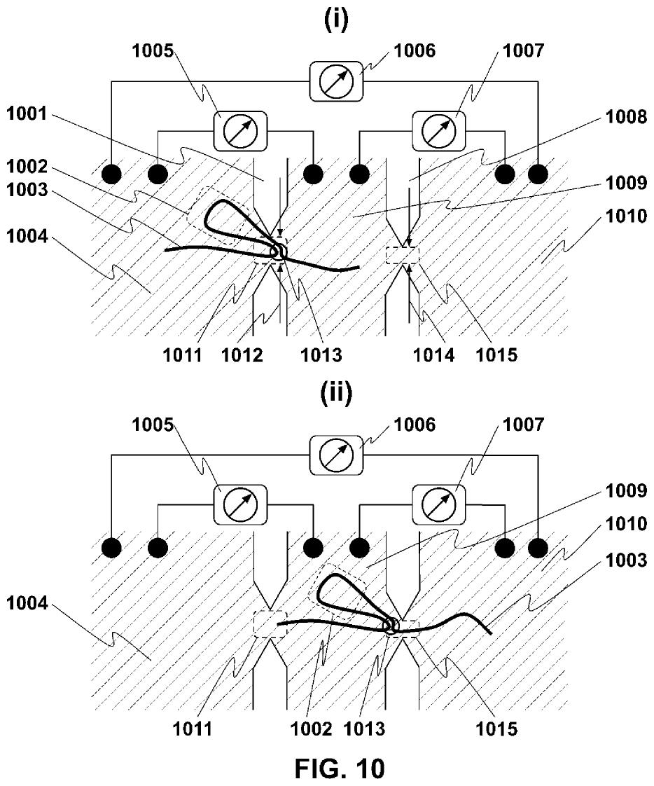

In another embodiment, shown in FIG. 10, there are least two constriction regions fluidically connected in series with each other, such that the at least two constriction regions have a different property. In some embodiments, the different property is a different sized cross-section. In the preferred embodiment, the smallest dimensional length of the cross-section, the constriction device's “critical dimension”, reduces in a monotonic manner through the serial fluidic connection. In the preferred embodiment, the cross section of the constriction region is designed to either pass through, block, or physically alter a long nucleic acid molecule with a structure from fully translocating said constriction region for a certain minimum translocation force or below. As demonstrated in FIG. 10, (i) a long nucleic acid molecule (1003) with a structure (1013) is translocated through, and interrogated by, a first constriction region (1011) of a constriction device (1001) of critical dimension (1012). In this drawn embodiment, there is a second constriction region (1015) from a second constriction device (1008) with a smaller critical dimension (1015) that is fluidically connected in series via a middle fluidic chamber (1009). In some embodiments, the translocation of the molecule through the first constriction region is controlled by the SMU (1005) in electrical contact with the entrance fluidic chamber (1004) and middle fluidic chamber (1009), or by the SUM (1006) in electrical contact with the entrance fluidic chamber (1004) and the exit fluidic chamber (1010), or a combination of both. Upon fully translocating the first constriction region, the molecule is then (ii) at least partially translocated through, and interrogated by, a second constriction region (1011). In this drawn embodiment, the molecule is unable to fully translocate the second constriction region with the applied translocation force due to the critical dimension (1014) of the second constriction region being too narrow to accommodate the structure (1013) on the long nucleic acid molecule (1003). In some embodiments, the translocation of the molecule through the second constriction region is controlled by the SMU (1007) in electrical contact with the middle fluidic chamber (1009) and exit fluidic chamber (1010), or by the SUM (1006) in electrical contact with the entrance fluidic chamber (1004) and the exit fluidic chamber (1010), or a combination of both.

In some embodiments, the long nucleic acid molecule with a structure is only able to fully translocate a constriction region with a certain critical dimension by increasing the translocation force applied on the molecule. In some embodiments, the translocation force required to fully translocate a particular molecule with a structure in a particular physical configuration through a constriction region is repeatable measurement for a constriction device with a particular cross-sectional shape and critical dimension of the constriction region.

In the preferred embodiments, the interrogation of the at least one structure on the long nucleic acid molecule by the at least two constriction devices, each with a different property, such that the two devices respectively generate a signal when interrogating said structure, and the comparative analysis of the two signals can be analyzed to determine a property of the structure.

In some embodiments, the at least two constriction devices have two different critical dimensions. In some embodiments, the first constriction region of a first constriction device has a critical dimension that is at least 10% larger than a second constriction region of a second constriction device, or at least 25% larger, or at least 50% larger, or at least 100% larger, or at least 150% larger, or at least 200% larger. In some embodiments, the at least two constriction devices have two different cross-section geometries. For example, one constriction region is oval in shape with the oval's major axis about 15 nm in diameter, and the minor axis about 5 nm in diameter, while the second constriction is circular in shape, about 10 nm in diameter. In some embodiments, the length of the critical dimension along the center axis of the constriction region is different between the at least two constriction regions. For example, the first constriction region has a critical dimension that is 5 nm in length along the central axis, and the second constriction region as a critical dimension that is 15 nm in length along the central axis.

In some embodiments, there is an additional fluidic connection to the middle fluidic chamber (1009). In some embodiments, this middle fluidic chamber allows for the entry, or exit, of a long nucleic acid molecule into the middle fluidic chamber without translocating through a constriction device. In the preferred embodiment, the fluidic connection is used to exit a long nucleic molecule with at least one structure, whose at least one structure is unable to translocate through the second constriction region. In some embodiments, the conditions in the middle chamber can be altered via fluidic connection, for example: pH, reagent composition, reagent concentration, ionic conditions. In some embodiments, the reagent comprises enzymes, labeling bodies, or nucleotides.

In some embodiments, at least a portion of the long nucleic acid molecule may be located within the constriction region of one constriction device, while at least a second portion of said molecule is located within the constriction region of a second constriction device. In some embodiments, both said constriction devices are interrogating their respective portions of long nucleic acid molecule simultaneously.

In some embodiments, the different property of the at least two different constriction devices is a surface energy property of at least a portion of the constriction regions.

In some embodiments, the different property of the at least two different constriction devices is a surface functionalization property of at least a portion of the constriction regions.

In some embodiments, the different property of the at least two different constriction devices is the type of an enzyme bound directly or indirectly to the surface of at least a portion of the constriction regions.

For brevity, in this drawn embodiment (FIG. 10), the constriction devices are of the current blockade constriction device of the type previously described for FIG. 3(A) in the definition of “constriction device”. However, the other previously described constriction devices described can also be used as variations of this embodiment method, with similar variation in the constriction region critical dimension between the constriction regions in series.

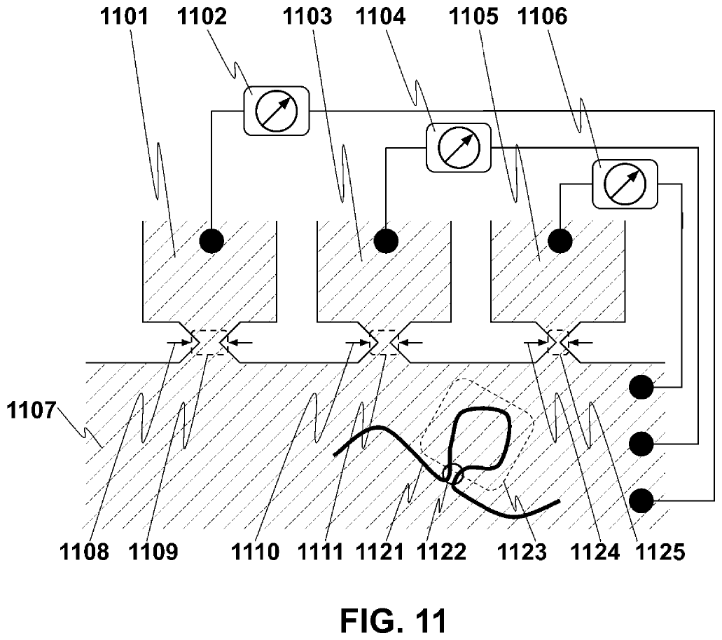

In another embodiment, shown in FIG. 11, there are least two constriction regions fluidically connected via an originating fluidic chamber (1107), such that the at least two constriction regions have a different property. In some embodiments, the property is the constriction region cross-section. In the preferred embodiment, a long nucleic acid molecule (1121) with a structure (1122) is introduced into the originating fluidic chamber (1107) via fluidic connection (not shown), such that the molecule is presented with at least two constriction devices, each of which comprises a different property. In the drawn embodiment the property is the critical dimension and there are three constriction devices: a first constriction region (1109) of a first constriction device with an associated critical dimension (1108) in which the molecule translocation is controlled by an SMU (1102) into a first exit fluidic chamber (1101), a second constriction region (1111) of a second constriction device with an associated critical dimension (1110) in which the molecule translocation is controlled by an SMU (1104) into a second exit fluidic chamber (1103), and a third constriction region (1125) of a third constriction device with an associated critical dimension (1124) in which the molecule translocation is controlled by an SMU (1106) into a third exit fluidic chamber (1105).

In the preferred embodiment, the molecule is interrogated by each constriction region in a sequential and selective manner. In some embodiments, the order of interrogation is from smallest critical dimension to largest. In some embodiments, the order of interrogation is from largest critical dimension to smallest. In some embodiments, the order of interrogation is from nearest to farthest. In some embodiments, the order of interrogation is random. In some embodiments, the order of interrogation is based on a sensing profile of each constriction region. In some embodiments, the molecule is interrogated by only a sub-set of the constriction regions. In some embodiments, the molecule is interrogated by at least one constriction region multiple times.

In some embodiments, the molecule is specifically collected at a desired output fluidic chamber such that the molecule can be sorted from other molecule.

This device embodiment is particularly advantageous for solid state devices where-by the constriction region is defined by a manufacturing process, for example: a semiconductor manufacturing process. Such a process will have a process variation of constriction region critical dimensions and cross-section shapes. Here, the process variation of the manufacturing process can be used to generate multiple different devices, which are then characterized for their physical profile after or during manufacture. This information can then be used by a control system to select the sub-set and order of the constriction regions to be used for interrogation. In some embodiments, the different constriction region geometries are randomly assigned by manufacturing process variation. In some embodiments, the different constriction region geometries are purposely assigned by manufacture design. In some embodiments, the different constriction region geometries are assigned by a combination of random manufacturing process variation and controlled design.

In some embodiments the property that differentiates the at least two constriction devices is a baseline measurement of a control by said constriction devices. In some embodiments, the control consists of constriction device interrogating an unoccupied constriction region, in that only a conductive liquid solution is present in the constriction region during the measurement. In some embodiments, the control consists of a known macromolecule, or a known un-labelled nucleic acid molecule, or known nucleic acid molecule with at least one known bound labelling body, or a known nucleic acid molecule with at least one known structure.

For embodiments where-by the constriction device comprises a biological pore, a mixture of different biological pores can be used during the constriction device assembly process, and after assembly into a constriction device, have their respective pore dimensions characterized to determine their absolute or relative size with respect to each other.

For some embodiments the multiple constriction devices are separated from each other by at least 50 nm, or by at least 100 nm, or by at least 500 nm, or by at least 1000 nm, or by at least 5 microns, or by at least 10 microns, or by at least 50 microns, or by at least 100 microns, or by at least 500 microns.

In some embodiments, at least a portion of the long nucleic acid molecule may be located within the constriction region of one constriction device, while at least a second portion of said molecule is located within the constriction region of a second constriction device. In some embodiments, both said constriction devices are interrogating their respective portions of long nucleic acid molecule simultaneously.

In some embodiments, the different property of the at least two different constriction devices is a surface energy property of at least a portion of the constriction regions.

In some embodiments, the different property of the at least two different constriction devices is a surface functionalization property of at least a portion of the constriction regions.

In some embodiments, the different property of the at least two different constriction devices is the type of an enzyme bound directly or indirectly to the surface of at least a portion of the constriction regions.

For all embodiments whereby there are at least two constriction devices, in some embodiments the fluidic chamber that fluidically connects the at least two constriction devices is physically configured such that distance between at least one pair of constriction devices is about the physical length of a single structure. In some embodiments, about the physical length of two structures. In some embodiments, about the physical length of three structures.

For all embodiments whereby there are at least two constriction devices, in some embodiments the fluidic chamber that fluidic connects the at least two constriction devices can have the solution modified in said chamber. In some embodiments, the modification is an addition of a reagent, a change in reagent concentration, a change in solution composition, a change in solution ionic conductivity or a change in solution pH. In some embodiments, the regent is a digestive enzyme.

In some embodiments, the fluidic device comprises the electrodes. In some embodiments, the electrodes are silver chloride electrodes.

For all embodiments whereby there are at least two constriction devices, in some embodiments a single SMU can be used to measure between a multiple of electrode pairs. This is accomplished by including a switching network to allow for the system control to select which pair of electrodes to measure from. For example, the measure the ion current through a first SMU, or a second SMU, or both the first and the second SMU. In some embodiments, at least a portion of the switching network is external to the fluidic device. In some embodiments, the fluidic device comprises at least a portion of the switching network. For example, the fluidic device may include a network work addressable transistors that allows for selection of electrode pairs.

For brevity, in this drawn embodiment (FIG. 11), the constriction devices are of the current blockade constriction device of the type previously described for FIG. 3(A) in the definition of “constriction device”. However, the other previously described constriction devices described can also be used as variations of this embodiment method, with similar variation in the constriction region critical dimension between the constriction regions.

In another embodiment wherein a blocking current constriction device of that shown in FIG. 3(A) is used to interrogate a long nucleic acid molecule, a retarding force is applied on at least a portion(s) of the molecule, such that said force opposes the translocation force applied on the molecule in the constriction region. In some embodiments, the retarding force opposes the translocation force via a natural response to the movement of the long nucleic acid molecule, when said molecule moves due to a translocation force. For example: a frictional force. In some embodiments, the retarding force is an external force applied on at least a portion of the molecule that opposes the translocation force. In some embodiments the external force is controlled via control system. In some embodiments, the control system uses a feedback system, in which at least one input parameter comprises a signal from the constriction device. In some embodiments, the control system uses a feedback system, in which at least one input parameter comprises data from fluorescently interrogating said long nucleic acid molecule.

Typically a current blocking constriction device operates by translocating the molecule through the constriction with the same force that drives the sensing current through the constriction region. As a consequence, halting the molecule translocation results in no current, and thus no constriction device signal. Furthermore reducing the translocation speed of the molecule results in a reduced current, and thus a reduced constriction device signal strength, which may result in the signal falling below the system noise floor. As such, a long nucleic acid molecule cannot be simultaneously interrogated while halted or moving below a certain threshold translocation speed. With this limitation, certain features of interest along the molecule, for example a labelling body or structure, cannot be selectively interrogated over a desired range of different currents. In this embodiment, a retarding force is added to slow, or stop, or reverse the molecule's movement through the constriction region for a certain sensing current driving force, when compared to the translocation speed of the same molecule, in the same constriction region, with the same current driving force, with no retarding force applied. With such an embodiment, the translocation speed, and the driving force of the sensing current can be de-coupled.

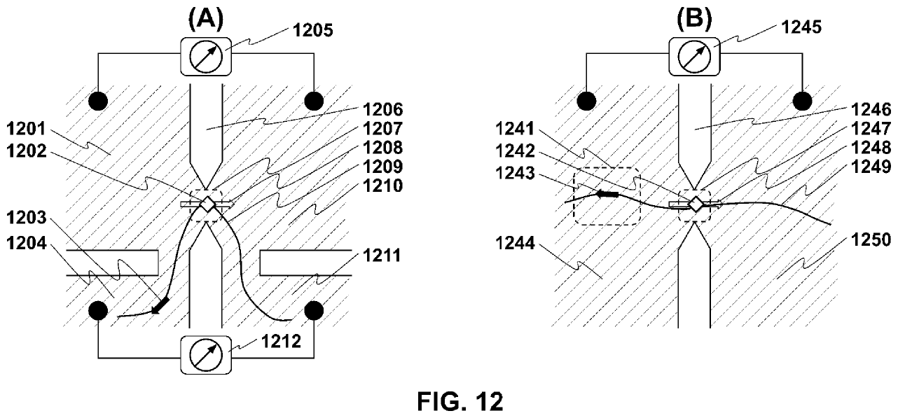

The FIG. 12(A) demonstrates an example device and method embodiment wherein there is one retarding fluidic channel (1204) in fluidic connection with the input fluidic chamber (1201), and there is one collection fluidic channel (1211) in fluidic connection with the output fluidic chamber (1210), such that the constriction device (1206) fluidically connects the input fluidic chamber and outlet fluidic chamber through the constriction region (1207). In this drawn embodiment, within the retarding fluidic channel there is a retarding force (1203) that opposes the translocating force (1208). Furthermore, in this drawn embodiment there are two SMUs, a first SMU (1205) and a second SMU (1212). Both SMUs can be used together, or independently to translocate the molecule through the constriction region. In the preferred embodiment, the second SMU (1212) is used to bring the molecule from the retarding fluidic channel, through the constriction, into the collection fluidic channel, while doing so, allowing for interrogation of the molecule in the constriction region via the current blockade, said current driven and sensed by the second SMU. In such a manner, there exists a translocation force along the entire length of the molecule as an electrical field is applied between the electrodes originating from the second SMU. In the preferred embodiment, the second SMU is used to translocate the molecule through the constriction region until a feature of interest (1202) is identified. Once identified, the second SMU is then electrically disconnected, and the feature of interest is then interrogated in the constriction region with the first SMU, wherein the first SMU is used to drive and sense the current through the constriction region. In such an embodiment, the majority of the translocation force acting on the molecule from the first SMU driven current will be largely applied to the region of the molecule in the constriction region, furthermore, the portion(s) of the molecule in the retarding fluidic channel and collection fluidic channel will be largely uninfluenced from the first SMU. In addition, a retarding force (1203) in the retarding channel will oppose the translocation force, slowing or halting the molecule's movement through the constriction region during the interrogation with the first SMU (1205). With such an embodiment, the feature of interest can be interrogated with a higher sensing current, and at a lower translocation speed, when compared to a system with no such retarding force, thus allowing for a large range of constriction currents while interrogating of the feature of interest, including its physical shape, physical conformation, physical configuration, or physical composition.

In some embodiments, there is no collection fluidic channel (1211), only an output fluidic chamber (1210). In some embodiments, there is no retarding fluidic channel (1204), only an input fluidic chamber (1201).

In some embodiments, the current through the constriction region is modulated while the feature of interest is at least partially maintained inside the constriction region. In some embodiments, the current through the constriction region is modulated while the feature of interest is translocating through the constriction region with a translocation speed reduced by a retarding force. In some embodiments, the modulation of the current is controlled by a feedback system in which at least one input to the system is a measurement of the current through the constriction region. In the preferred embodiment, the current is modulated so as to optimize the signal-to-noise ratio of the interrogation of the feature of interest.

In some embodiments, a coordinated control process is used to operate the two SMUs such one SMU positions the at least a portion of the feature of interest in the constriction region, while at least a second SMU is used to interrogate the at least a portion of the feature of interest in the constriction region. In the preferred embodiment, when one SMU is operating, the other SMU is electrically disconnected.

In some embodiments, the collection fluidic channel is also a retarding fluidic channel such that if the translocation force (1208) is reversed, a retarding force can be applied on the portion(s) of the long nucleic acid in the collection fluidic channel that opposes the reversed translocation force.

In some embodiments the SMU(s) (1205 and 1212) operate simultaneously. In some embodiments, they operate separately. In some embodiments, when one SMU is operating, the other SMU is electrically disconnected.

In some embodiments, as shown in FIG. 12(B), the is no retarding fluidic channel, but rather a retarding region (1241) within the input fluidic chamber (1244) from which a retarding force (1243) can be applied on at least portion(s) of the long nucleic acid molecule (1249) to oppose the translocation force (1248) in the constriction region (1247) of the constriction device (1246) originating from the SMU (1245). In some embodiments, there is a retarding region within the output fluidic chamber (1250) such that if the translocation force (1248) is reversed, a retarding force can be applied on at least portion(s) of the long nucleic acid in the output fluidic chamber that opposes the reversed translocation force.

In some embodiments, the features of interest comprises a structure, or a specific sequence, or bound label body, or a gene, or a promoter region, or an enhancer region, or a loop, or specific physical map pattern, or an undefined or unknown entity associated with a constriction device signal.

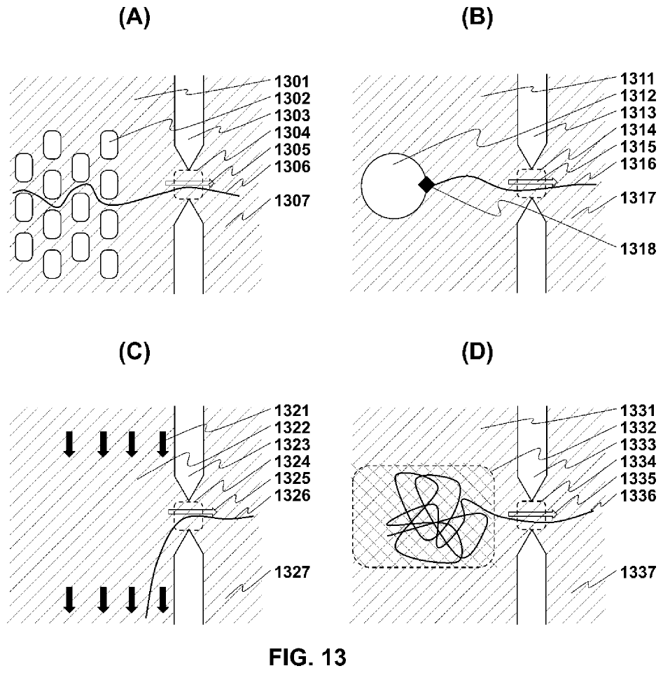

FIG. 13(A) demonstrates a retarding force that comprises a shear or frictional force generated from the interaction of the long nucleic acid molecule (1306) with fluidic features (here patterned fluidic features that include pillars (1302)) that opposes the translocation force (1305) applied on the molecule in the constriction region (1304) of the constriction device (1303).

In some embodiments, the fluidic features comprises patterned fluidic features. In the preferred embodiment, the patterned fluidic features have a separation distance of less then 10 microns, more preferably less than 5 microns, even more preferably less than 2 microns. All types of pillar sizes, shapes, and density, and pitch, and spacing are possible for this embodiment. In some embodiments the pillars are ovals, or rectangles, or diamonds, or squares, or random shapes. In some embodiments the pillars are arranged in an ordered manner. In some embodiments the pillars are arranged in a random order. In some embodiments, the fluidic feature comprises physical obstacles. In some embodiments, fluidic feature comprises a channel, or a collection of channels. In some embodiments, the pathway along which the long nucleic acid molecule navigates through the fluidic features comprises at least one sharp corner with a >45 degree turn, or preferably >90 degree turn, or more preferably >110 degree turn, so as to maximize the interaction of the long nucleic acid molecule with the surface of the fluidic features. In some embodiments there is at least 1 turn along a 50 micron length pathway, or preferably at least 2 turns along a 50 micron length pathway, or more preferably, at least 5 turns along a 50 micron length pathway.

In some embodiments, the fluidic features comprises a porous material. In some embodiments, the porous material comprises a gel.

In some embodiments, the fluidic features comprises at least one bead, nano-particle, or microbead.

In some embodiments, the magnitude of the retarding force has a monotonically increasing relationship with the length of the portion of the long nucleic acid molecule in the retarding region. In some embodiments, this relationship is approximately linear.

FIG. 13(B) demonstrates a retarding force that comprises a drag force, or a pulling, or a holding force generated by at least one chemical bond (1318) of the long nucleic acid molecule (1316) to a physical body or functionalized surface region (1312), such that the retarding force opposes the translocation force (1315) applied on the molecule in the constriction region (1314) of the constriction device (1313). In some embodiments, the input fluid chamber (1311) comprises the body. In some embodiments, the body is a bead, a dendrimer, or a quantum dot. In some embodiments, the body is tip of a contact probe, for example an atomic force microscope. In some embodiments, the body is a macromolecule. In some embodiments, the body's physical position relative to the constriction device can be modulated. In some embodiments, this modulation is via an electrical-mechanical system, or a pressure driven system, or a deformable system, or a phase-change material, or a piezoelectric system.

In some embodiments, the retarding force comprises a frictional or shear force generated by a region within the fluidic device whereby at least one confining dimension of the fluidic chamber is less than 100 nm, preferably less than 50 nm, more preferably less than 30 nm. For example, a fluidic channel or chamber wherein the height of the fluidic channel or chamber is 30 nm. Here the height of the channel or chamber provides a confining dimension in which the long nucleic acid molecule physically interacts with the floor and the ceiling, and thus is capable of generating a frictional or shear force to counter a translation force.

FIG. 13(C) demonstrates a retarding force that comprises a shear force generated by fluid flow (1321) within the fluidic device. In the embodiment drawn in FIG. 13(C), a fluid flow is present on at least one side of the constriction device (1323) such that fluid flow generates a shear force on the long nucleic acid molecule (1326). In some embodiments, a fluid flow rate may be 0.1 microns/s or greater, or 1 microns/s or greater, or 2 microns/s or greater, or 5 microns/s or greater, or 10 microns/s or greater, or 25 microns/s or greater, or 100 microns/s or greater, or 250 microns/s or greater, or 1000 microns/s or greater

FIG. 13(D) demonstrates a retarding force that comprises an entropic energy minimization force generated by a region (1332) within the inlet fluidic chamber (1331) that together with inlet fluidic chamber comprises an entropic barrier to the long nucleic acid that is at least partially occupying said region, such that said molecule will experience a force pulling it into said region. For background on entropic barriers, and the force applied on long nucleic acid molecules by such barriers, refer to application number: PCT/US21/34754. Said force will be a retaining force opposing the translocation force (1335) applied on the molecule in the constriction region (1314) of the constriction device (1313).

In some embodiments, various combinations of retarding forces are applied on the long nucleic acid molecule.

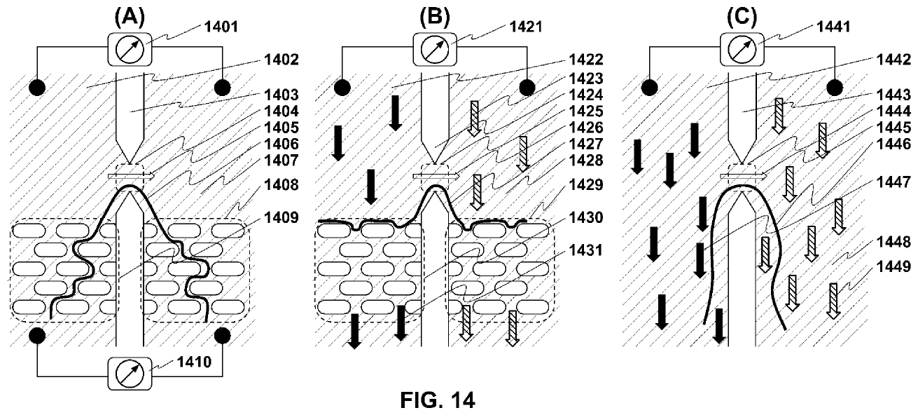

FIG. 14(A) demonstrates an embodiment wherein the retarding force is provided by a porous material. In this particular drawn embodiment, the porous material is a patterned collection of pillars (1409 and 1408) on either side of the constriction device (1403). In this particular drawn embodiment, a frictional or shear force is generated on the long nucleic acid molecule (1406) by the porous material (1409) to oppose the movement of the molecule by the translocation force (1405) applied on the molecule, with said translocation force generated by the first SMU (1401) driving the sensing ionic current through the constriction region (1404) of the constriction device, where in this particular drawing, the ionic current is flowing one conductive solution fluidic chamber (1407) to the other conductive solution fluidic chamber (1402). In addition, a secondary SMU (1410) can be used to move the long nucleic acid molecule both through the porous material and constriction region. In some embodiments, the porous material is only present on one side of the constriction device. In the preferred embodiment, the porous material is on both sides such that a retarding force is present regardless of the orientation of the translocation force. In some embodiments, the secondary SMU is used to position a particular feature or region of interest within the constriction region. In the preferred embodiments, the two SMUs operate under the control of a feedback system in which at least one input parameter comprises a signal from the constriction device. In some embodiments, the two SMUs operate under the control of a feedback system in which at least one input parameter comprises data from fluorescent interrogation of the long nucleic acid molecule.

FIG. 14(B) demonstrates an embodiment wherein the retarding force is provided by the long nucleic acid molecule being pushed with an applied force against a fluidic feature. In this particular drawn embodiment, the fluidic feature is a porous material, and the applied force is a fluid flow. Here, on both sides of the constriction device (1424) there is a porous material (1430 and 1429), and on both sides of the constriction device is a fluid flow (1431 and 1423) into the respective porous materials. In this particular drawn embodiment, a frictional or shear force is generated on the long nucleic acid molecule (1427) by the contact of the porous material (1430) and said molecule, with said force opposing the movement of the molecule by the translocation force (1426) applied on said molecule, with said translocation force generated by the SMU (1421) driving the sensing ionic current through the constriction region (1425) of the constriction device, where in this particular drawing, the ionic current is flowing one conductive solution fluidic chamber (1428) to the other conductive solution fluidic chamber (1422). In some embodiments, a porous material or fluid flow is only present on one side of the constriction device. In the preferred embodiment, a porous material and fluid flow is present on both sides such that a retarding force is present regardless of the orientation of the translocation force. In some embodiments the fluid flow rates on both side are the same. In some embodiments, the fluid flow rate on both sides are different. In the preferred embodiments, the SMU and at least one fluid flow rate (1431 or 1423) operate under the control of a feedback system in which at least one input parameter comprises a signal from the constriction device. In some embodiments, the SMU and at least one fluid flow rate (1431 or 1423) operate under the control of a feedback system in which at least one input parameter comprises data from fluorescent interrogation of the long nucleic acid molecule.

FIG. 14(C) demonstrates an embodiment wherein the retarding force is provided by a shear force applied on the long nucleic acid molecule from a fluid flow in which at least a portion of said molecule is exposed. In this particular drawn embodiment, on both sides of the constriction device (1443) there is a fluid flow (1447 and 1449), with each fluid flow resulting in an independent shear force acting on said molecule. In this particular drawn embodiment, each shear force applied to the long nucleic acid molecule (1446) is independent of the translocation force (1445), in that, unlike a frictional force which opposes movement of said molecule (for example, a movement caused by the translocation force), each shear force applied on the molecule is a function of a fluid flow rate, the fluid properties, and the portion of the molecule within said fluid flow. In this particular drawn embodiment, there are two shear forces acting on the long nucleic acid molecule, one shear force originating from the portion of the long nucleic acid molecule exposed to one fluidic flow (1447), and a second shear force originating from the portion of the long nucleic acid molecule exposed to a second fluidic flow (1449). Here, at least one shear force is used to oppose the movement of the molecule by the translocation force (1445) applied on the molecule, with said translocation force generated by the SMU (1441) driving the sensing ionic current through the constriction region (1444) of the constriction device, where in this particular drawing, the ionic current is flowing one conductive solution fluidic chamber (1448) to the other conductive solution fluidic chamber (1442). In some embodiments, a fluid flow is only present on one side of the constriction device. In some embodiments, a fluid flow is present on both sides of the constriction device. In some embodiments the fluid flow rates on both sides are the same. In some embodiments, the fluid flow rate on both sides are different. In the preferred embodiments, the SMU and at least one fluid flow rate (1447 or 1449) operate under the control of a feedback system in which at least one input parameter comprises a signal from the constriction device. In some embodiments, the SMU and at least one fluid flow rate (1447 or 1449) operate under the control of a feedback system in which at least one input parameter comprises data from fluorescent interrogation of the long nucleic acid molecule.

For all embodiments, the long nucleic acid molecule can include at least one labeling body bound to at least one structure. In some embodiments, the labeling body is fluorescent. In some embodiments, the labeling body is specific to a particular structure, or a particular complex, or to a particular protein. In some embodiments, there may be more than one type of labelling, in which each type has a different fluorescent property. In some embodiments, the different type of fluorescent property is used to identify a different specific binding target. In some embodiments, the spatial data of the fluorescent interrogation during a certain time period is coordinated with at least one signal obtained from the constriction device at during the same time period. In the preferred embodiment, the fluorescent data can be used to identify a property of the structure present in the constriction region when said structure is being interrogated by the constriction device. In some embodiments, the property is a protein type, or a complex type.

For all embodiments, the translocation of the molecule through the constriction region can be stopped, started, reversed, and have the speed adjusted on-the-fly. In some embodiments, a feedback mechanism is used to control the translocation velocity or force. In some embodiments, the feedback mechanism uses the constriction signal as at least one input parameter. In some embodiments, the feedback mechanism uses a fluorescent signal as at least one input parameter.

For all embodiments, the long nucleic acid molecule can include bound labelling bodies capable of generating a physical map when interrogated by the constriction device, or a fluorescent imaging device. In some embodiments, the physical map is a feature density physical map. In some embodiments, the physical map is an AT/CG density physical map. In some embodiments, the long nucleic molecule is interrogated by a constriction device under conditions suitable to partially melt the molecule. In some embodiments, the fluorescent interrogation occurs during a time point in which said molecule is also being interrogated by a constriction device. In the preferred embodiment, the fluorescent labeling bodies also provide for a linear physical map that can be interrogated by the constriction device. In the preferred embodiment, the spatial fluorescent linear physical map is interrogated a multitude of times by the fluorescent interrogation device, and the time point of each data set can be coordinated with the time point of the constriction device. In some embodiments, such coordination allows for a registration of where along the major axis of the long nucleic molecule (with respect to the fluorescent linear physical map) a measured signal with the constriction device is taken. In some embodiments, the fluorescent data allows for a determination of the long nucleic acid molecule's velocity at a particular time point of the constriction device interrogation. The velocity may be the global (average) speed of the molecule's mass, or the particular translocation speed of the portion of the molecule in the constriction device, or both. In some embodiments, the fluorescent data allows for a determination of the long nucleic acid molecule's stretch at a particular time point of the constriction device interrogation. The stretch may be the global (average) stretch (extension) of the molecule, or the particular stretch of the portion of the molecule in the constriction device, or both. All such data can be used to provide contextual location information to the constriction data, or to signal process the constriction device data, or both. For example, the fluorescent data may provide information as to proximity to a particular gene, or promoter region during the measurement of a constriction device signal. Or, the fluorescent data can be used to correct for a variation in translocation speed of the long nucleic acid molecule through the constriction device as a function of time.

### EXAMPLES

### Example 1: AT/CG Feature Density Physical Mapping with a Constriction Device

As an initial proof of concept, DNA with a feature density linear physical map is prepared for interrogation with a current blockade constriction device of the type previously described in Figure A(A). In this example, the physical map comprises a long nucleic acid molecule labelled with intercalating molecules along the length of the molecule, prepared as a melt map, such that the density of the intercalating molecules bound along the length of the long nucleic acid molecule correlates with the CG content of the long nucleic acid molecule as was previously described for molecule 521 in FIG. 5.

Human genomic DNA is isolated from blood samples by embedding purified nuclei in low melting point agarose plugs [Zhang, 2012]. The sample is electroeluted into low salt denaturing buffer (0.1× TBE, 20 mM NaCl, 2% β-mercaptoethanol) with YOYO-1 at a ratio of 1 dye per 10 nucleotide pairs and incubated at 18 C overnight. The sample is diluted 1:1 with formamide with minimal manipulation and heated to 31 C for 10 minutes [Tegenfeldt, 2009, U.S. Pat. No. 10,434,512] before quenching on ice.

The intended constriction device lateral geometries are first defined using a CAD software program such that the large fluidic feature (>5 micron) contact photomasks can be specified for order from a mask vendor, while the smaller features electronically transferred to an electron beam lithography (EBL) system for direct writing. First, a glass borofloat wafer 0.5 mm thick is patterned with chrome/gold alignment markers using a photolithography and metal lift-off process, to be used for registration of all subsequent patterning. Next, an ELB resist (ZEP-520A) is spin coated onto the glass wafer to the manufacturer's instructions, and exposed to a focused electron beam lithography system, to write the constriction region aligned to the metallic alignment marks. The pattern is developed with N-amyl acetate and etched using CF4 plasma to a depth of about 10 nm in the constriction region (the larger features around the constriction region will etch deeper, approximately to a depth of 20 nm), followed by removal of resist using NMP. The EBL writing and etching process defines the constriction dimensions, which are then confirmed with scanning electron microscopy. The final pore size is about 10 nm in diameter.

Next, the same glass borofloat wafer is spin coated with a layer of positive photoresist, and then prepared for exposure according to the resist manufactures instructions. Operating a mask aligner in contact mode, aligned to the metal alignment marks, the resist on the wafer is exposed through the mask to UV light, after which the resist is developed according to the instructions and chemicals recommended by the manufacturer to remove the exposed resist from the glass substrate and expose the glass surface in the fluidic channels that connect both sides of the constriction device. The exposed glass is then etched in reactive ion etcher using a CHF3 plasma to etch 1000 nm deep. The resist is then removed in an oxygen ash plasma.

With both the constriction region and fluidic connection channel now patterned in the surface of the glass substrate, the channels ends are connected to ports by sand blasting through the glass wafer using a metal shadow mask. The metallic alignment markers are then etched away in a solution etchant, and the glass substrate is then thoroughly washed in a heated mixture of water, ammonia, and hydrogen peroxide to remove any remaining organic material and facilitate particle removal from the surface. Finally, the fluidic device is completed by plasma assisted fusion bonding the patterned glass wafer to a non-patterned glass wafer at 400 C, and then annealed in an oven at 650 C. Once cooled, the wafer is then diced into individual chips, and the fluidic ports are interfaced with a plastic manifold allowing for luer lock connections to all inlet and outlet ports.

The sample solution is then introduced to device on both sides of the constriction device, and Ag/AgCl electrodes are inserted to the buffer to apply voltage and measure current. The current and voltage signal is collected by Molecular Device Multi-Clamp 700B, and digitized by Axon Digidata 1550. The captured signal is then processed and filtered to identify the time point at which a long nucleic acid molecule enters and exits the constriction device, wherein the data collected between those time points represents the raw signal trace of the molecule in question. This data is then further processed and filtered to identify current blockade associated with a bound intercalating molecule. Using look up tables and reference data sets of known control molecules, both labeled and unlabeled, the molecule data is converted to an AT melt map profile binned at 100 bp, wherein each bin represents the proportion of labels within the 100 bp bin normalized to an average bin value determined from a collection of interrogated molecules.

The interrogated molecule is then compared with a reference to identify the molecule within a known human genomic reference. The pre-computed reference physical maps are derived from sequences of the human genome assembly GRCh37 analyzed for melting state by the method of [Tostesen, 2005]. Reference map segments are sampled at intervals corresponding to bins of 100 bp, with each bin worth of GC ratio information is normalized as a signed 8 bit integer, where −128 represents 100% AT, 127 represents 100% GC. The reference map is pre-computed for a variety (up to 20) DNA translocation velocities, so the same sequence is present multiple times. Observed maps are compared with the physical map references in two steps, first each molecule is artificially segmented into 32 bin segments starting every other bin. The dot product of each segment and a 32 bin tile of the reference map segments is computed. The top 4 k matches are passed to the second stage, which repeats the dot product on neighboring regions in both the map and the sample and scores them with a Smith-Waterman algorithm to permit local insertions and deletions. Detection cutoffs are determined empirically.

### Example 2: Interrogating a Higher Order Nucleic Acid Structure with a Parallel Multi-Pore Constriction Device

As an initial proof of concept, a long nucleic acid molecule with a higher order nucleic acid structure is prepared for interrogation with a multi-constriction device. As (B-cell lymphoma) cells were cultured are cultured in RPMI 1640 medium supplemented with 10% fetal bovine serum and 1% serum at 39° C. in 5% CO2 in air, progressing during cell cycle from G1/G2 interphases, with more stretched genomic DNA towards more condensed prophase, prometaphases, metaphases forms. The metaphase chromosomes could be prepared using typical conditions of 100 ng/ml Colcemid for 2.5 h 75 mM KCl for 5 min Me/Ac fixation drop/dry on slides Vectashield with DAPI and image quality control by imaging using a cooled CCD or SiCMOS camera on a wide-field microscope with a 100× NA 1.4 Plan Apochromat lens and analyzed by typical image softwares such as softWoRx by Applied Precision. To prepare more stretched interphase DNA, with G2 arresting, Doxycycline (BD) dissolved in water (1 mg/ml) is added to a final concentration of 0.5 μg/ml, 1NM-PP1 dissolved in DMSO (10 mM) is added to cultures at a final concentration of 2 μM. Degradation of AID-containing proteins is induced by addition of a 50 mM solution of Indole-3-acetic acid (auxin, Fluka) dissolved in ethanol to a final concentration of 125 μM. To prevent cells from entering anaphase, Nocodazole (Sigma-Aldrich) dissolved in DMSO at 1 mg/ml is added to some cultures to a final concentration of 0.5 μg/ml. For chromosomal length measurements for image data control, pictures are taken for each condition using microscope and analysed using IMARIS.

Single cell samples can be flow sorted. Cells are suspended overnight in ice-cold 70% ethanol. The next morning, cells are rinsed with PBS then re-suspended in PBS containing 100 μg/ml RNase A and 5 μg/ml propidium iodide. Samples are then analyzed using a FACSCalibur flow cytometer following the manufacturer's instructions. Data is analyzed using FlowJo V10.3. Cells are gated for viability based on forward and side scatter (FSC/SSC), from which single cells are selected based on FSC height (H) and width (W).

Chromosome conformation capture is performed as follows: 10-20×106 cells are cross-linked in 1% formaldehyde for 10 minutes and quenched in 125 mM glycine. Cells are snap-frozen and stored at −80° C. before cell lysis. Cells are lysed for 15 minutes in ice cold lysis buffer (10 mM Tris-HCl pH8.0, 10 mM NaCl, 0.2% Igepal CA-630) in the presence of Halt protease inhibitors (Thermo Fisher, 78429) and cells are disrupted by homogenization with pestle A for 2×30 strokes. Chromatin is solubilized in 0.1% SDS at 65° C. for 10 minutes, quenched by 1% Triton X-100 (Sigma, 93443).

At the higher compaction stage of prometaphase, the chromosome/chromatin presents a linear density at 50-70 Mb/μm (micron) of the radius of scaffold at 30 to 100 nm. The height of one helical turn to be ˜200 nm in late prometaphase, which is also the size of the layer (12-Mb layer at a linear density of 60 Mb/mm) suggesting consecutive genomic loci follow a helical gyre. In prophase, condensin II compacts chromosomes into arrays of consecutive loops and sister chromatids split along their length. Upon nuclear envelope breakdown and entry into prometaphase, condensin II-mediated loops become increasingly large as they split into smaller ˜80-kb loops by condensin I. Chromosomes are shown as arrays of loops. During prometaphase, the nested arrangements of centrally located condensin II-mediated loop bases and more peripherally located condensin I-mediated loop bases are the central scaffold acquires a helical arrangement with loops rotating around the scaffold as steps in a spiral staircase. As prometaphase progresses, outer loops grow, the number of loops per turn increases, and chromosomes shorten to form the mature mitotic chromosome. with a pitch of ˜250 nm within the cylindrical shape of chromatids (Gibcus et al., Science 359, 6135 (2018) 9 Feb. 2018).

The intended fluidic device that contains 3 current blockade constriction regions is fabrication in a manner similar to that described in Example 1. However, in this example, 3 distinct constriction devices, each with its own current blockade constriction region, are designed in a similar layout to the device shown in FIG. 11. In this example, all 3 constriction devices are fluidically connected to an originating fluidic chamber (1107). Patterning of the 3 distinct constriction regions is performed by an EBL system, wherein the critical dimension of the respective restriction regions are patterned with an average electron beam dose (245 μC/cm2) with a dosage compensation profile for critical dimensions that was previously calibrated to pattern nanopores of approximately 10 nm-500 nm in diameter. The originating fluidic chamber is connected to an inlet port, while the 3 constriction devices are each fluidically connected to their own separate outlet port. The 3 constriction devices are physically separated from each by a spacing of 50 microns.

After the device is fabricated, the entire device is wetted with a conducting solution (2 M LiCl, 10 mM Tris, 1 mM, EDTA, pH=8.8 buffer), and each constriction device is electrically connected with its own respective SMU (1102, 1104, and 1106) for characterization as shown in FIG. 11. As with the previous example, the SMUs are Molecular Device Multi-Clamp 700B, and digitized by Axon Digidata 1550. Each constriction device is then characterized for its electrical properties, with said properties used to determine the respective constriction region's absolute and relative physical profiles. Characterization involves measuring the ion current through a constriction region while the constriction region has a +/−100 mV triangle wave applied, over a frequency range of 0.01 to 100 kHz. After characterization it is determined that constriction region 1 (1109) has a critical dimension of 50 nm, constriction region 2 (1111) has a critical dimension of 150 nm, and constriction region 3 (1125) has a critical dimension of 300 nm. Based on this analysis, it is desired to interrogate a molecule from smallest to largest constriction region critical dimension.

After the device has been characterized, an input sample is introduced into the originating fluidic chamber (1107). Using the first SMU (1102) associated with the 50 nm constriction region (1109), the molecule is electrokinetically driven towards the region with an applied voltage of 100 mV, and while doing so, the ion current through the constriction region (1109) is monitored. The molecule is registered at the constriction region when a sustained reduction in the measured current is observed from the baseline, indicating the molecule is present, and stuck, in the constriction region, thus indicating a substantial amount of higher order structure is present. The applied voltage is then increased in 50 mV steps to 500 mV, at each time monitoring the current, and comparing to the baseline, to confirm the molecule is still present in the constriction region, after which the voltage polarity is reversed to eject the molecule back into the originating fluidic chamber (1107). The first SMU (1102) is then disconnected, and the third SMU (1106) associated with the 150 nm constriction region (1125) repeats the process, however this constriction device is successfully able to completely translocate the molecule at an applied voltage of 300 mV. The current trace recorded during the translocation event is used to estimate chromatin fiber density by inferring the cross-sectional area of the chromatin strand as a function of linear position along the length of the fiber. The chromatin fiber density data are compared against a lookup table of known molecule profiles in order to map the fiber. Statistical distributions of the chromatin fiber density are recorded in order to assess the state of compaction and accessibility of the chromatin.

What is claimed is:

1. A method for analyzing a long nucleic acid molecule, comprising: (a) partially de-naturing at least a portion of said long nucleic acid molecule by exposing at least a portion of the molecule to at least one denaturing condition; (b) translocating at least a portion of said long nucleic acid molecule between a first conductive liquid medium and a second conductive liquid medium through at least one constriction region of at least one constriction device; (c) interrogating at least one signal associated with the at least one constriction device as the nucleic acid molecule interacts with the at least one constriction region of said at least one constriction device; and (d) determining a binned denaturing profile along at least a portion of the long nucleic acid molecule from said at least one signal.

2. The method of claim 1 wherein an ion current through the constriction region is measured to generate the signal.

3. The method of claim 1 wherein the at least one constriction device comprises an electrode gap of sufficient proximity to the constriction region of the device such that the long nucleic acid molecule translocating through said constriction region also translocates between said electrode gap, such that an electrical measurement can be performed to generate the signal.

4. The method of claim 1 wherein the at least one constriction device comprises a sensor of sufficient proximity to the constriction region of the device such that said molecule translocating through said constriction region will be sensed by the sensor, generating the signal.

5. The method of claim 4 wherein the sensor comprises a transistor.

6. The method of claim 4 wherein the sensor comprises a functionalized surface.

7. The method of claim 1 wherein the constriction of the constriction device is tangible.

8. The method of claim 1 wherein the constriction of the constriction device is intangible.

9. The method of claim 1 wherein the signal is captured in the constriction region of the constriction device.

10. The method of claim 1 wherein the signal is captured in proximity to the constriction region of the constriction device.

11. The method of claim 1 wherein the signal generated from the portion of the partially melted long nucleic acid molecule is measurably different than a signal that would have resulted from the same portion of said molecule in a fully hybridized state.

12. The method of claim 1 wherein the denaturing condition comprises a temperature.

13. The method of claim 1 wherein the denaturing condition comprises a reagent.

14. The method of claim 1 wherein the denaturing condition comprises an ionic strength.

15. The method of claim 1 wherein the denaturing condition comprises a pH.

16. The method of claim 1 wherein the denaturing condition is modulated.

17. The method of claim 16 wherein the denaturing condition is modulated during the interrogation.

18. The method of claim 16 wherein the denaturing condition is modulated between multiple interrogation events of said molecule.

19. The method of claim 16 wherein the denaturing condition is modulated to increase uniqueness of the binned denaturation profile of at least a portion of said long nucleic acid molecule.

20. The method of claim 16 wherein the modulation is controlled by a feedback system in which at least one input parameter is the signal from said constriction device.

21. The method of claim 1 wherein a first side of the constriction region has a first denaturing condition and a second side of the constriction region has a second denaturing condition, and wherein the first denaturing condition and the second denaturing condition are different.

22. The method of claim 1 wherein at least a portion of said long nucleic acid molecule is interrogated by said constriction device a plurality of time.

23. The method of claim 22, wherein said plurality of interrogations are used to generate a consensus binned denaturation profile.

24. The method of claim 1 wherein the binned denaturation profile constitutes a linear physical map.

25. The method of claim 24 comprising comparing said linear physical map to a reference.

26. The method of claim 25 wherein a variation relative to said reference indicates a structural variation in the long nucleic acid molecule relative to the reference.

27. The method of claim 25 wherein said comparing is used to identify information associated with a disease.

28. The method of claim 25 wherein this comparing is used to identify at least a portion of the long nucleic acid molecule.

29. The method of claim 28 wherein identifying the at least a portion of the long nucleic acid molecule comprises assigning an originating organism, class, species, ethnicity, family genealogy, individuals, tissues, cells, chromosome, phase, variant, gene, or location within a genome to the long nucleic acid molecule.

30. A method for analyzing higher order nucleic acid structure of a long nucleic acid molecule, comprising: (a) translocating at least a portion of said long nucleic acid molecule between a first conductive liquid medium and a second conductive liquid medium through at least one constriction region of at least one constriction device; (b) interrogating at least one signal associated with the at least one constriction device as the long nucleic acid molecule translocates through the at least one constriction region of said at least one constriction device; and (c) determining a property of said structure from said at least one signal.

31. The method of claim 30 wherein an ion current through said constriction region is measured to generate the signal.

32. The method of claim 30 wherein the at least one constriction device comprises an electrode gap in proximity to the constriction region such that the long nucleic acid molecule translocating through said constriction region will also translocate through said electrode gap, such that an electrical measurement can be performed to generate the signal.

33. The method of claim 30 wherein the at least one constriction device comprises a sensor of sufficient proximity to said device's constriction region, such that said long nucleic acid molecule translocating through said constriction region will be sensed by the sensor, generating the signal.

34. The method of claim 33 wherein the sensor comprises a transistor.

35. The method of claim 33 wherein the sensor comprises a functionalized surface.

36. The method of claim 30 wherein the constriction of the constriction device is tangible.

37. The method of claim 30 wherein the constriction of the constriction device is intangible.

38. The method of claim 30 wherein the signal is captured in the constriction region of the constriction device.

39. The method of claim 30 wherein the signal is captured in proximity to the constriction region of the constriction device.

40. The method of claim 30 wherein the signal generated from the portion of the long nucleic acid molecule with a structure is measurably different than a signal that would have resulted from the same portion of said molecule without said structure.

41. The method of claim 30 wherein the higher order nucleic acid structure comprises a nucleosome.

42. The method of claim 30 wherein the higher order nucleic acid structure comprises a nucleosome clutch.

43. The method of claim 30 wherein the higher order nucleic acid structure comprises chromatin.

44. The method of claim 30 wherein the higher order nucleic acid structure comprises a chromatin nanodomain.

45. The method of claim 30 wherein the higher order nucleic acid structure comprises a CCCTC binding factor.

46. The method of claim 30 wherein the higher order nucleic acid structure comprises a loop.

47. The method of claim 30 wherein the higher order nucleic acid structure comprises a topologically associating domain.

48. The method of claim 30 wherein the higher order nucleic acid structure comprises a loop domain.

49. The method of claim 30 wherein the higher order nucleic acid structure comprises a compartment A.

50. The method of claim 30 wherein the higher order nucleic acid structure comprises a compartment B.

51. The method of claim 30 wherein the higher order nucleic acid structure comprises an enhancer-promoter complex.

52. The method of claim 30 wherein the higher order nucleic acid structure comprises an insulator complex.

53. The method of claim 30 wherein the higher order nucleic acid structure comprises a transcription factor complex.

54. The method of claim 30 wherein the higher order nucleic acid structure comprises a CTCF protein.

55. The method of claim 30 wherein the higher order nucleic acid structure comprises a PDS5 protein.

56. The method of claim 30 wherein the higher order nucleic acid structure comprises a WAPL protein.

57. The method of claim 30 wherein the higher order nucleic acid structure comprises a heterochromatin, a euchromatin, or a heterochromatin-euchromatin boundary.

58. The method of claim 30 wherein the higher order nucleic acid structure comprises a transcription factor.

59. The method of claim 30 wherein the higher order nucleic acid structure comprises a methyl-binding protein.

60. The method of claim 30 wherein the higher order nucleic acid structure comprises a chromatin remodeling protein.

61. The method of claim 30 wherein the higher order nucleic acid structure comprises a Histone deacetylase (HDAC).

62. The method of claim 30 wherein the higher order nucleic acid structure comprises a nucleic acid binding protein.

63. The method of claim 30 wherein the higher order nucleic acid structure comprises a regulatory factor binding protein.

64. The method of claim 30 wherein the higher order nucleic acid structure comprises a nucleic acid repair protein.

65. The method of claim 30 wherein the higher order nucleic acid structure comprises a telomere modification protein.

66. The method of claim 30 wherein the higher order nucleic acid structure comprises a repeat region binding protein.

67. The method of claim 30 wherein the higher order nucleic acid structure comprises a ribonucleic acid (RNA), small interfering RNA (siRNA), micro RNA (miRNA), guide RNA (gRNA), Long non-coding RNA (lncRNA).

68. The method of claim 30 wherein the higher order nucleic acid structure comprises a nucleoprotein complex.

69. The method of claim 30 wherein the higher order nucleic acid structure comprises a CRISPR Cas9 complex.

70. The method of claim 30 wherein the higher order nucleic acid structure comprises an argonaut complex.

71. The method of claim 30 wherein the higher order nucleic acid structure comprises a cohesin associated loop.

72. The method of claim 30 wherein the higher order nucleic acid structure comprises a condensin associated loop

73. The method of claim 30 wherein at least one sequence-specific labeling body is bound to said long nucleic acid molecule.

74. The method of claim 30 wherein the property of the said structure comprises information associated with a disease.

75. The method of claim 74 wherein the disease is a cancer.

76. The method of claim 30 wherein the property of said structure comprises physical size of the structure.

77. The method of claim 30 wherein the property of said structure comprises physical orientation with respect to a long axis of said long nucleic acid molecule.

78. The method of claim 30 wherein the property of said structure comprises flexibility of the structure.

79. The method of claim 30 wherein the property of said structure comprises a number of loops contained within.

80. The method of claim 30 wherein the property of said structure comprises a length of at least one loop contained within.

81. The method of claim 30 wherein the property of said structure is interrogated using at least two different translocation forces.

82. The method of claim 30 wherein the property of said structure is interrogated using at least two fluidically connected constriction devices, each having a different constriction region property.

83. The method of claim 82 wherein the constriction region property comprises a cross-section.

84. The method of claim 82 wherein the constriction region property comprises a critical dimension.

85. The method of claim 82 wherein the constriction region property comprises a baseline un-occupied measured constriction device signal for fixed measurement condition.

86. The method of claim 82 wherein the constriction region property comprises a baseline measured constriction device signal when interrogating a known control molecule or macromolecule.

87. The method of claim 82 wherein the constriction region property comprises a surface energy.

88. The method of claim 82 wherein the constriction region property comprises translocation length.

89. The method of claim 82 wherein the constriction region property comprises surface functionalization.

90. The method of claim 82 wherein a selection mechanism is used to determine the order in which the at least two constriction devices will be used for interrogation.

91. The method of claim 90 wherein a selection mechanism is at least partially based a previous interrogation of said molecule.

92. The method of claim 90 wherein a selection mechanism is at least partially based on a constriction region property.

93. The method of claim 82 wherein the minimum translocation force on said long nucleic acid molecule necessary to translocate said molecule through said two constriction devices is different.

94. The method of claim 82 wherein a property of the solution fluidically connecting the two constriction devices can be modified while the long nucleic acid is in contact with the solution.

95. The method of claim 94 wherein the property comprises a reagent concentration.

96. The method of claim 95 wherein the reagent is a digestive enzyme.

97. The method of claim 94 wherein the property comprises an ionic concentration.

98. The method of claim 94 wherein the property comprises a pH, a conductivity, a density, or a viscosity.

99. The method of claim 94 wherein the modification of the solution property is used to modify the physical conformation of said higher order nucleic acid structure.

100. The method of claim 30 wherein the long nucleic acid molecule is bound with at least two labeling bodies of one label body type.

101. The method of claim 100 wherein the said labeling bodies constitute a physical map.

102. The method of claim 100 wherein said labelling bodies can be interrogated by said constriction device.

103. The method of claim 100 wherein said labelling bodies can be interrogated by a fluorescent interrogation device.

104. The method of claim 103 wherein the fluorescent interrogation is done while at least a portion of said long nucleic acid molecule is being interrogated by at least one of the at least two constriction devices.

105. The method of claim 30 wherein the long nucleic molecule is at least partially in a partially melted state while being interrogated by one of the at least two constriction devices.

106. The method of claim 105 wherein said partially melted state constitutes a physical map.

107. The method of any one of claim 101 or 106 wherein said physical map is compared to a reference.
